(19) United States
(12) Patent Application Publication
(10) Pub. No.: US 2024/0060985 A1
(43) Pub. Date: Feb. 22, 2024

(54) SYSTEMS AND METHODS FOR ANALYTE ANALYSIS

(71) Applicant: COMBINATI INCORPORTATED, Carlsbad, CA (US)

(72) Inventors: Hien NGUYEN, San Jose, CA (US); Ju-Sung HUNG, Carlsbad, CA (US); Lingxia JIANG, Boyds, MD (US)

(21) Appl. No.: 18/269,896

(22) PCT Filed: Dec. 28, 2021
(86) PCT No.: PCT/US2021/065361

§ 371 Date: Jun. 27, 2023

Related U.S. Application Data
(60) Provisional application No. 63/173,038, filed on Apr. 9, 2021.
(60) Provisional application No. 63/131,473, filed on Dec. 29, 2020.

(51) Int. Cl. G01N 33/68; C12Q 1/6804; G01N 33/53; G01N 33/543

(52) U.S. Cl. CPC G01N 33/6803; C12Q 1/6804; G01N 33/5308; G01N 33/54386

(57) ABSTRACT

The present disclosure provides methods and systems for analysis of an analyte. Analysis of an analyte may include contacting an analyte with a binding reagent, which binding reagent may include a binding probe with specificity for the analyte and a nucleic acid molecule. The nucleic acid molecule may be denatured and signals indicative of the denaturing of the analyte may be collected. The collected signals may be used to detect, identify, or quantify the analyte.

### CROSS-REFERENCE

This application claims the benefit of both U.S. Provisional Patent Application No. 63/131,473, filed Dec. 29, 2020, and U.S. Provisional Patent Application No. 63/173,038, filed on Apr. 9, 2021, the contents of which are entirely incorporated herein by reference.

### BACKGROUND

Microfluidic devices are devices that contain structures that handle fluids on a small scale. Typically, a microfluidic device operates on a sub-millimeter scale and handles micro-liters, nano-liters, or smaller quantities of fluids. One application of microfluidic devices is in analyte analysis, e.g., digital polymerase chain reaction (dPCR). Microfluidic devices with multiple partitions may be useful for dPCR. Unlike quantitative real-time PCR (qPCR) where templates are quantified by comparing the rate of PCR amplification of an unknown sample to the rate for a set of known qPCR standards, dPCR may provide higher sensitivity, better precision, and greater reproducibility.

For genomic researchers and clinicians, dPCR is particularly powerful in rare mutation detection, quantifying copy number variants, and Next Gen Sequencing library quantification. The potential use in clinical settings for liquid biopsy with cell free DNA and viral load quantification further increases the value of dPCR technology. Existing dPCR solutions have used elastomeric valve arrays, silicon through-hole approaches, and microfluidic encapsulation of droplets in oil. Despite the growing number of available dPCR platforms, dPCR has been at a disadvantage when compared to the older qPCR technology which relies on counting the number of PCR amplification cycles. The combination of throughput, ease of use, performance and cost are the major barriers for gaining adoption in the market for dPCR.

### SUMMARY

Provided herein are methods and systems that may be useful for detecting, identifying, or quantifying an analyte or multiple analytes. The present disclosure provides methods, systems, and devices for sample preparation, nucleic acid amplification, analyte analysis, multiplex analyte analysis, or any combination thereof. The method, systems, and devices described herein can permit detection, identification, or quantification of analytes at a reduced cost or complexity as compared to other systems and methods.

In an aspect, the present disclosure provides methods for identifying an analyte, comprising: (a) contacting a binding reagent with the analyte, which binding reagent comprises:
(i) a binding probe having binding specificity for the analyte, and (ii) a nucleic acid molecule; (b) denaturing at least a portion of the nucleic acid molecule or derivative thereof; and (c) detecting signals indicative of the denaturing to identify the analyte.

In some embodiments, the analyte is coupled to a support. In some embodiments, the analyte is coupled to the support via a capture agent immobilized on the support. In some embodiments, the capture agent is an antibody. In some embodiments, the binding reagent is an antibody. In some embodiments, the analyte is a protein.

In some embodiments, the analyte is one of a plurality of analytes in a sample, and wherein the binding reagent is one of a plurality of binding reagents. In some embodiments, the method further comprises contacting an additional binding reagent with an additional analyte, which the additional binding reagent comprises: (i) an additional binding probe having binding specificity for the additional analyte, and (ii) an additional nucleic acid molecule. In some embodiments, the additional nucleic acid molecule is different than the nucleic acid molecule. In some embodiments, the method further comprises denaturing the additional nucleic acid molecule or derivative thereof. In some embodiments, the method further comprises detecting additional signals indicative of the denaturing the additional nucleic acid or derivative thereof to identify the additional analyte. In some embodiments, the method further comprises partitioning the plurality of binding reagents into a plurality of partitions.

In some embodiments, the method further comprises using a microfluidic device to partition the plurality of binding reagents into the plurality of partitions. In some embodiments, the method further comprises imaging one or more partitions of the plurality of partitions to detect the signals. In some embodiments, the method further comprises determining a number of partitions which contain the nucleic acid molecule, wherein the number of partitions which contain the nucleic acid molecule are used to quantify the analyte.

In some embodiments, the nucleic acid molecule is reversibly coupled to the binding probe. In some embodiments, the method further comprises, prior to (b), decoupling the nucleic acid molecule from the binding probe. In some embodiments, the method further comprises, prior to (b), decoupling a portion of the nucleic acid molecule from the binding probe. In some embodiments, subsequent to (a), the binding reagent is coupled to the analyte. In some embodiments, the method further comprises removing uncoupled binding reagent by washing. In some embodiments, the method further comprises subjecting the nucleic acid molecule to controlled heating to denature the nucleic acid molecule. In some embodiments, the method further comprises, prior to (b), amplifying the nucleic acid molecule. In some embodiments, the method further comprises processing the signals to generate a denaturation profile, which denaturation profile is used to identify the analyte. In some embodiments, the nucleic acid molecule comprises an intercalating dye from which the signals are derived. In some embodiments, the signals are optical signals.

In some embodiments, the method further comprises contacting an additional binding reagent with the analyte, which additional binding reagent comprises: (i) an additional binding probe having binding specificity for the analyte, and (ii) an additional nucleic acid molecule. In some embodiments, the method further comprises contacting the nucleic acid molecule and the additional nucleic acid molecule with a splinter oligo to couple the nucleic acid molecule to the additional nucleic acid molecule. In some embodiments, the method further comprises ligating the nucleic acid molecule and the additional nucleic acid molecule to generate a ligated nucleic acid molecule. In some embodiments, the method further comprises, prior to (b), amplifying the ligated nucleic acid molecule.

In another aspect, the present disclosure provides system for identifying an analyte, comprising: a detection unit configured to collect and process signals for identification of the analyte; one or more processors operatively coupled to the detection unit, wherein the one or more processors are individually or collectively programmed or otherwise configured to: (i) contact a binding reagent with the analyte, which binding reagent comprises (a) a binding probe having binding specificity for the analyte, and (b) a nucleic acid molecule; (ii) denature at least a portion of the nucleic acid molecule or derivative thereof; and (iii) direct the detection unit to detect signals indicative of denaturing of the nucleic acid molecule to identify the analyte.

In some embodiments, the system further comprises a support configured to couple to the analyte. In some embodiments, the support comprises a capture agent immobilized on the support, and wherein the capture agent is configured to couple to the analyte. In some embodiments, the capture agent is an antibody. In some embodiments, the binding reagent is an antibody. In some embodiments, the analyte is a protein.

In some embodiments, the system further comprises a microfluidic device comprising a plurality of partitions. In some embodiments, the plurality of partitions is configured to partition a mixture comprising the binding reagent. In some embodiments, the detection unit is configured to image one or more partitions of the plurality of partitions. In some embodiments, the system further comprises a processing unit configured to amplify the nucleic acid molecule. In some embodiments, the system further comprises a heating unit configured for controlled heating of the nucleic acid molecule to denature the nucleic acid molecule. In some embodiments, the one or more processors are individually or collectively programmed or otherwise configured to generate a denaturation profile of the nucleic acid molecule, which denaturation profile is usable to identify the analyte.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example workflow for analyzing an analyte;

FIG. 2 schematically illustrates an example complex comprising an analyte, capture agent, and binding reagent;

FIG. 3 schematically illustrates an example workflow for analyzing an analyte;

FIGS. 4A and 4B illustrate example denaturation profiles for multiple binding reagents; FIG. 4A illustrates example denaturation profiles for example samples contacted with a single binding reagent; FIG. 4B illustrates example denaturation profiles for example samples contacting with multiple binding reagents;

FIG. 5 schematically illustrates example identification and quantification of example analytes;

FIG. 6 schematically illustrates an example computer control system that is programmed or otherwise configured to implement methods provided herein;

FIGS. 7A-C illustrate an example proximity ligation assay (PLA) workflow for use with digital polymerase chain reaction (PCR); FIG. 7A schematically illustrates an example proximity ligation assay and nucleic acid amplification; FIG. 7B schematically illustrates an example proximity ligation assay without subsequent nucleic acid amplification; FIG. 7C illustrates an example proximity ligation assay integrated with digital PCR;

FIG. 8 schematically illustrates an example multi-plate and multi-sample PLA with digital PCR workflow;

FIG. 9 illustrates an example standard curve of interleukin 6 protein quantification;

FIG. 10 illustrates an example standard curve of interleukin 6 protein quantification with background correction;

FIG. 11 illustrates an example standard curve of troponin protein quantification with background correction;

FIG. 12 illustrates an example digital melt analysis using an intercalating dye;

FIG. 13 schematically illustrates an example using PLA and digital PCR with digital melt analysis with an intercalating dye; and

FIG. 14 schematically illustrates another example PLA workflow.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the term “binding reagent” generally refers to a species comprising one or more moieties (e.g., a binding probe) with affinity for or capable of binding to an analyte and can also include one or more other moieties, including an identifier molecule or barcode. The identifier or barcode can identify one or more moieties with affinity for or capable of binding to an analyte, and, thus, the analyte to which the one or more moieties bind. A binding reagent may comprise a nucleic acid molecule, antibody, or any other molecule capable of binding to the analyte. A binding reagent may include a binding probe and a nucleic acid molecule identifier or barcode. Binding reagents may be present in solution. Binding reagents may couple to analytes immobilized on a support. Binding reagents may couple to analytes immobilized on a support via a capture agent. Alternatively, or in addition to, binding reagents may couple to analytes present in a solution.

As used herein, the term “binding probe” generally refers to a species, such as a nucleic acid molecule (e.g., aptamer), antibody, or any other molecule which has specificity for an analyte or a domain of an analyte. A binding probe may comprise a moiety, domain, or structure that is capable of coupling to (e.g., binding to) or that couples to an analyte. A binding probe may reversibly couple to or bind to an analyte. Alternatively, a binding probe may irreversibly couple to or bind an analyte. A binding probe may be coupled to an identifier molecule or barcode (e.g., nucleic acid molecule), to form a binding reagent. The binding reagent may be usable for identifying an analyte. A binding probe may be reversibly or irreversibly (e.g., covalently) coupled to an identifier molecule or barcode. In an example, the binding probe is coupled to a nucleic acid configured as an identifier molecule or barcode.

As used herein, the term “capture agent” generally refers to a molecule coupled to a support and capable of binding to or coupling with an analyte, where such coupling or binding immobilizes the analyte to the support. A capture agent molecule may be a ligand, nucleic acid molecule, protein, antibody, or any other molecule capable of capturing an analyte. A capture agent may have a specificity for a single analyte or type of analyte. Alternatively, or in addition to, a capture agent may have a specificity for or may bind to multiple analytes or multiple types of analytes.

The term “sample,” as used herein, generally refers to any sample containing or suspected of containing an analyte. For example, a sample can be a biological sample containing one or more analytes. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is obtained from a cell-free bodily fluid, such as whole blood. In such instance, the sample can include cell-free DNA, cell-free RNA, proteins, metabolites, or any combination thereof. In some examples, the sample can include circulating tumor cells, cancer biomarkers, or both. In some examples, the sample is an environmental sample (e.g., soil, waste, ambient air and etc.), industrial sample (e.g., samples from any industrial processes), and food samples (e.g., dairy products, vegetable products, and meat products). The sample may be processed prior to loading into a microfluidic device. For example, the sample may be processed to lyse cells, purify proteins, or to include reagents. Alternatively, or in addition to, the sample may not be processed prior to loading into a microfluidic device.

As used herein, the term “nucleic acid” refers to polymers of nucleotides or derivatives thereof. As used herein, the term “target nucleic acid” refers to a nucleic acid that is desired to be amplified in a nucleic acid amplification reaction. For example, the target nucleic acid may comprise a nucleic acid template. In some embodiments, the target nucleic acid may be the product of the ligation of at least two oligonucleotides to one another.

As used herein, the term “fluidic” or “microfluidic” may be used interchangeable and generally refer to a chip, area, device, article, or system including at least one channel in fluid communication with an array of chambers. The channel may have a cross-sectional dimension less than or equal to about 10 millimeters (mm), less than or equal to about 5 mm, less than or equal to about 4 mm, less than or equal to about 3 mm, less than or equal to about 2 mm, less than or equal to about 1.5 mm, less than or equal to about 1 mm, less than or equal to about 750 micrometers (μm), less than or equal to about 500 μm, less than or equal to about 250 μm, less than or equal to about 100 μm, or less. The chambers may have a volume of less than or equal to about 100 microliters (μL), 50 μL, 25 μL, 10 μL, 5 μL, 1 μL, 500 nanoliters (nL), 250 nL, 100 nL, 50 nL, 25 nL, 10 nL, 5 nL, 1 nL, 500 picoliters (pL), 250 pL, 100 pL, 50 pL, 25 pL, 10 pL, 5 pL, 1 pL, or less.

As used herein, the term “fluid,” generally refers to a liquid or a gas. A fluid cannot maintain a defined shape and will flow during an observable time frame to fill the container into which it is put. Thus, the fluid may have any suitable viscosity that permits flow. If two or more fluids are present, each fluid may be independently selected among any fluids (e.g., liquids, gases, and the like).

As used herein, the term “partition,” generally refers to a division into or distribution into portions or shares. For example, a partitioned sample is a sample that is isolated from other samples. Examples of structures that enable sample partitioning include wells, chambers, droplets, or any combination thereof.

As used herein, the terms “pressurized off-gassing” or “pressurized degassing” may be used interchangeable and generally refer to removal or evacuation of a gas (e.g., air, nitrogen, oxygen, etc.) from a channel or chamber of the device (e.g., microfluidic device) to an environment external to the channel or chamber through the application of a pressure differential. The pressure differential may be applied between the channel or chamber and the environment external to the channel or chamber. The pressure differential may be provided by the application of a pressure source to one or more inlets to the device or application of a vacuum source to one or more surfaces of the device. Pressurized off-gassing or pressurized degassing may be permitted through a film or membrane covering one or more sides of the channel or chamber.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The methods, systems, and devices described herein may permit detection, identification, and quantification of multiple analytes (e.g., proteins) in a sample without complex and expensive multiplex consumables. Additionally, methods, systems, and devices described herein may permit reliable and accurate protein detection and quantification of proteins without reliance on inaccurate and difficult to reproduce protein standard curves. In another example, the binding probe is coupled to a nucleic acid that is the product of a ligation reaction and configured as an identifier molecule or barcode.

### Methods for Identifying Analytes

In an aspect, the present disclosure provides for methods for identifying or detecting analytes. The methods may comprise contacting a binding reagent with an analyte. The binding reagent may include a binding probe and a nucleic acid molecule. The binding probe may have binding specificity for the analyte such that the binding probe couples to or binds to the analyte. The nucleic acid molecule may include a molecule or sequence (e.g., identifier or barcode) that corresponds to or is usable for identifying or detecting the analyte. The method may further include denaturing the nucleic acid molecule or a derivative thereof or a portion of the nucleic acid molecule or a derivative thereof. Denaturing the nucleic acid or derivative thereof may generate detectable signals. The nucleic acid or a derivative thereof may be a product of a ligation reaction between two or more nucleic acids. In some cases, the nucleic acid or derivative thereof is the product of a ligation reaction between two different nucleic acids conjugated to or attached to two different binding reagents such as, for example, two different antibodies. The detectable signals may permit detection, identification, quantification, or any combination thereof of the analyte.

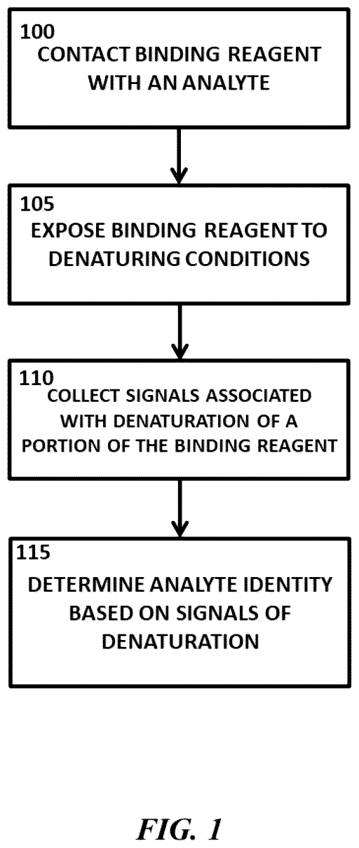

An example method for identifying an analyte is shown in FIG. 1. The example method may include contacting a binding reagent with an analyte 100 in a sample. The sample may include a single analyte or multiple analytes. A binding reagent may include a binding probe and a nucleic acid molecule, both of which may be specific to an analyte. For example, a first binding reagent with a first binding probe and first nucleic acid molecule may bind to a first analyte. A second binding reagent with a second binding probe and second nucleic acid molecule may bind to a second analyte. The first binding reagent may not be capable of binding to the second analyte and vice versa. The first nucleic acid molecule and second nucleic acid molecule may be different and distinguishable from one another such that the first nucleic acid molecule and second nucleic acid molecule may be usable for detection and identification of the first and second analytes, respectively. The nucleic acid molecule (e.g., first and second nucleic acid molecules) may be exposed to denaturing condition (e.g., thermal denaturing, chemical denaturing, etc.) 105. Upon denaturing of the nucleic acid molecule, detectable signals may be generated. In an example, signals may be optically detectable fluorescence signals and denaturation of the nucleic acid molecule may increase or decrease the fluorescence signals. The signals associated with denaturation of the nucleic acid molecule may be collected 110 and processed to identify the analyte 115.

The analyte may be provided as a part of a sample. A sample may be derived from a subject, such as a human, animal, or plant, or from soil, food, or other sample source. The sample may include a single analyte or may include multiple analytes. For example, the sample may include at least 1, 2, 3, 4, 5, 6, 8, 10, 15, 20 or more analytes. In an example, the sample includes at least 2 analytes. In another example, the sample includes at least 5 analytes. In another example, the sample includes at least 10 analytes. In another example, the sample includes at least 15 analytes. The analyte may be provided as part of a solution or may be immobilized on a support. The sample may include analytes of a same type or various types of analytes. For example, a sample may include proteins, nucleic acids, cells, metabolites, or other analytes. In an example, the analyte is a protein, protein fragment, or derivative thereof. The method may include detecting a single type of analyte (e.g., one or more different proteins) or multiple types of analytes (e.g., proteins and metabolites thereof). In an example, the method is usable for identification or quantification of one or more different proteins.

An analyte may be provided in a solution or coupled to a support. The analyte may be coupled to a support via a capture agent, covalently immobilized to the support (e.g., via a linker molecule), physically absorbed to a support, or any combination thereof. In an example, an analyte is provided in solution and the method includes coupling the analyte to a support. The analyte in solution may be brought into contact with a support comprising one or more capture agents. The capture agents may be immobilized on the support and may be usable for immobilizing the analyte onto the support. The capture agent may be a ligand, nucleic acid molecule, antibody, or any other molecule capable of binding to the analyte. The capture agent may have specificity for the analyte or may be a universal capture agent capable of binding multiple different analytes or different types of analytes (e.g., proteins, nucleic acid molecules, metabolites, etc.). In an example, the capture agent comprises an antibody and the analyte comprises a protein. In another example, the capture agent comprises an aptamer and the analyte comprises a protein, nucleic acid molecule, cell, metabolite, or other biological molecule. The capture agent may comprise a binding region that is specific to an analyte. Alternatively, or in addition to, the capture agent may comprise a binding region that is specific to a domain of an analyte, permitting the capture agent to bind to multiple different analytes.

The analyte may be contacted with a binding reagent. The binding reagent may comprise a binding probe and a nucleic acid molecule. The binding probe may comprise a ligand, nucleic acid molecule, antibody, or any other molecule capable of binding to the. In an example, the binding probe is an antibody and the binding reagent comprises an antibody. In another example, the binding probe is an aptamer and the binding reagent comprises an aptamer. The binding probe may have specificity for an analyte or portion of an analyte. The binding probe may not have specificity for another analyte or another portion of an analyte, thus, permitting the binding reagent to bind to a single analyte or type of analyte. The binding probe may permit reversible binding of the binding reagent to the analyte. Alternatively, or in addition, the binding probe may irreversibly bind the binding reagent to the analyte.

A binding reagent may include a nucleic acid molecule and a different binding reagent may include a different nucleic acid molecule. The nucleic acid molecule may be usable for detecting or identifying the analyte. For example, a first binding reagent may comprise a first nucleic acid molecule and may couple to a first analyte. A second binding reagent may comprise a second nucleic acid molecule and may couple to a second analyte. The first nucleic acid molecule and the second nucleic acid molecule may be different molecules. The differences in the first nucleic acid molecule and the second nucleic acid molecule may be detectable and, therefore, usable for identifying and distinguishing the first analyte from the second analyte. A sample may be contact with at least 1, 2, 3, 4, 5, 6, 8, 10, 15, 20, 30, 40, 50, or more binding reagents each coupled to at least 1, 2, 3, 4, 5, 6, 8, 10, 15, 20, 30, 40, 50, or more different nucleic acid molecules. A nucleic acid molecule may include a molecule or sequence (e.g., identifier or barcode) that is usable for identifying a given analyte.

The nucleic acid molecule may be a polymeric form of nucleotides of any length. For example, the nucleic acid molecule may include at least 1, 2, 3, 4, 5, 6, 8, 10, 15, 20, 30, 40, 50, 100, 500, 1000, or more nucleotides. The nucleotides may include deoxyribonucleotides, ribonucleotides, or analogs thereof. The nucleic acid molecule may comprise deoxyribonucleic acid (DNA), ribonucleic acid (RNA), peptide nucleic acid (PNA), lock nucleic acid (LNA), bridge nucleic acid (BNA), or any combination thereof. A nucleic acid may include one or more subunits selected from adenosine (A), cytosine (C), guanine (G), thymine (TO, and uracil (U), or variants thereof. A nucleotide can include A, C, G, T, or U, or variants thereof. A nucleotide can include any subunit that can be incorporated into a growing nucleic acid strand. Such subunit can be A, C, G, T, or U, or any other subunit that is specific to one of more complementary A, C, G, T, or U, or complementary to a purine (i.e., A or G, or variant thereof) or pyrimidine (i.e., C, T, or U, or variant thereof). In some examples, a nucleic acid may be single-stranded or double stranded, in some cases, a nucleic acid molecule is circular. A nucleic acid may comprise one or more modified nucleotides, such as methylated nucleotides and nucleotide analogs.

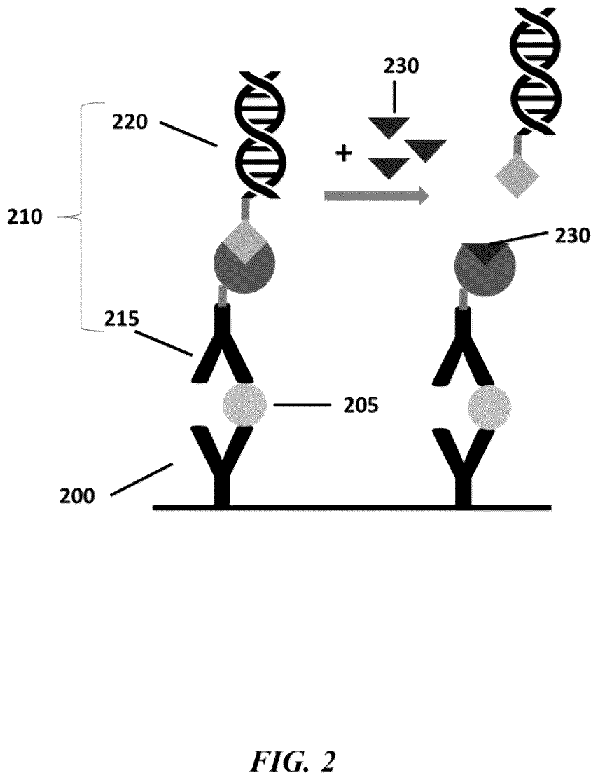

The method may include contacting a sample comprising at least one analyte with at least one binding reagent. In an example, a sample may have a plurality of analytes and may be contacted with a plurality of binding reagents. The analytes, or at least a portion of the analytes may be immobilized on a support and binding reagents with specificity for the analyte(s) may bind or couple to the analyte(s) to form a complex immobilized on the support. Other binding reagents (e.g., those with binding specificity for analytes not present in the sample) may not couple to the analytes and may remain in solution. The support and immobilized complex may be washed to remove unbound binding reagent. An example immobilized complex is shown in FIG. 2. The immobilized complex may include a capture agent 200 immobilized on the surface. The capture agent may be an antibody with specificity for an analyte 205. Alternatively, the capture agent 200 may be non-specific and may bind multiple analytes 205 or types of analytes (e.g., proteins). The capture agent 200 may immobilize the analyte 205 on a support (e.g., surface, bead, carrier, etc.). The complex may further include a binding reagent 210. The binding reagent may include a binding probe 215 with specificity for an analyte 205. In an example, the binding probe 215 may comprise or may be an antibody. In another example, the binding probe 215 may comprise or may be an aptamer. The binding reagent 210 may further include a nucleic acid molecule 220. The nucleic acid molecule 220 may be DNA, RNA, PNA, LNA, BNA, or any combination thereof. The nucleic acid molecule 220 may be double stranded or single stranded. In an example, the nucleic acid molecule 220 is double stranded. In another example, the nucleic acid molecule 220 is single stranded. The nucleic acid molecule 220 may be reversibly or irreversibly coupled to the binding probe 215. Alternatively, the nucleic acid molecule 220 may be covalently linked to the binding probe 215. In an example, the nucleic acid molecule 220 may be reversibly coupled to the binding probe 215 and the nucleic acid molecule may be disassociated by contacting the binding reagent with a molecule 230 with a higher affinity for the binding probe 215 than the nucleic acid molecule 220. The molecule 230 may displace the nucleic acid molecule 220 and bind to the binding probe 215, permitting the nucleic acid molecule 220 to enter the solution phase (e.g., no longer be bound to or immobilized on a support).

The method may further comprise identifying the nucleic acid molecule. Identification of the nucleic acid molecule may permit detection, identification, quantification, or any combination thereof of an analyte(s). The binding reagent (e.g., binding probe coupled to a nucleic acid molecule) may be analyzed to identify the nucleic acid molecule. Alternatively, or in addition to, the nucleic acid molecule or a portion of the nucleic acid molecule may be removed or cleaved from the binding reagent prior to identification or detection. The nucleic acid molecule may be reversibly coupled to the binding probe or may be irreversibly coupled to the binding probe. In an example, the nucleic acid molecule is reversibly coupled to the binding probe. In another example, the nucleic acid molecule is irreversibly coupled to the binding probe. A reversibly coupled nucleic acid molecule may be coupled to the binding probe by disulfide bonds, ligand binding, or other reversible bond. The reversibly coupled nucleic acid molecule may be decoupled or disassociate by contacting the binding reagent with a reducing agent, molecule with higher affinity for the binding probe, or any combination thereof. Alternatively, or in addition to, the nucleic acid molecule may be irreversibly coupled to the binding probe (e.g., covalently coupled). The nucleic acid molecule may be cleaved prior to analysis of the nucleic acid molecule. For example, the nucleic acid may include a consensus sequence for a restriction enzyme and the restriction enzyme may cleave the nucleic acid from the binding probe. Decoupling or cleaving of the nucleic acid molecule from the binding probe may permit the nucleic acid molecule to enter the solution phase while the analyte and binding probe remain immobilized on the support.

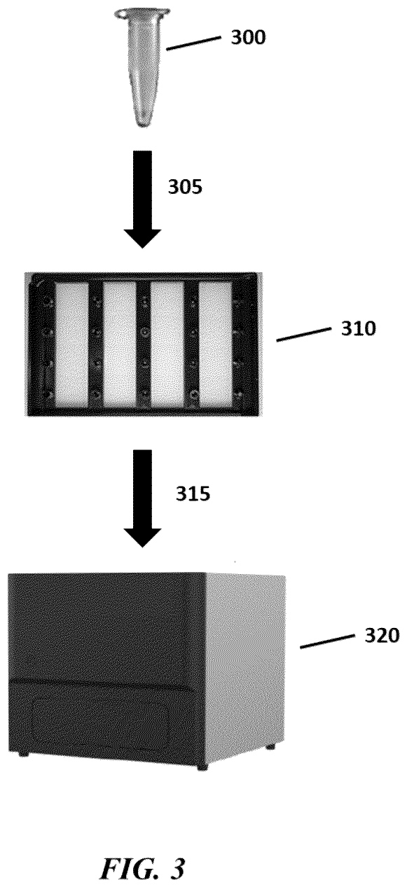

The nucleic acid molecule may be one of a plurality of nucleic acid molecule. The plurality of nucleic acid molecules may be provided to a fluidic device (e.g., microfluidic device). The microfluidic device may permit partitioning of the plurality of nucleic acid molecules. The nucleic acid molecules may be further processed (e.g., amplified) and subjected to denaturation conditions in the partitions. FIG. 3. Schematically illustrates a process flow for processing and analyzing the nucleic acid molecule. The nucleic acid molecule or plurality of nucleic acid molecules may be provided in a solution 300. The solution 300 may be directed to 305 a microfluidic device 310 via a fluid flow system comprising a pneumatic module or other fluid handling modules. The microfluidic device 310 may include a single array for partitioning the nucleic acid molecules or multiple arrays for partitioning the nucleic acid molecules. The microfluidic device may be loaded 315 into an analysis system 320. Alternatively, or in addition to, the analysis system 320 may include the microfluidic device 310 and the sample may be provided to the analysis system for partitioning 305 into the fluidic device 310.

The contacting of the binding reagent with said at least one analyte may include performing a proximity assay reaction (e.g. a proximity ligation assay (“PLA”) or a proximity extension assay (“PEA”), or a combination of both, for protein analysis (e.g., the detection of proteins analytes and/or protein interactions). PLA and PEA may use the conjugation of nucleic acid molecules to antibody probes (for instance, a polyclonal antibody or two monoclonal antibodies) having an affinity for a target protein analyte to detect the target protein analyte.

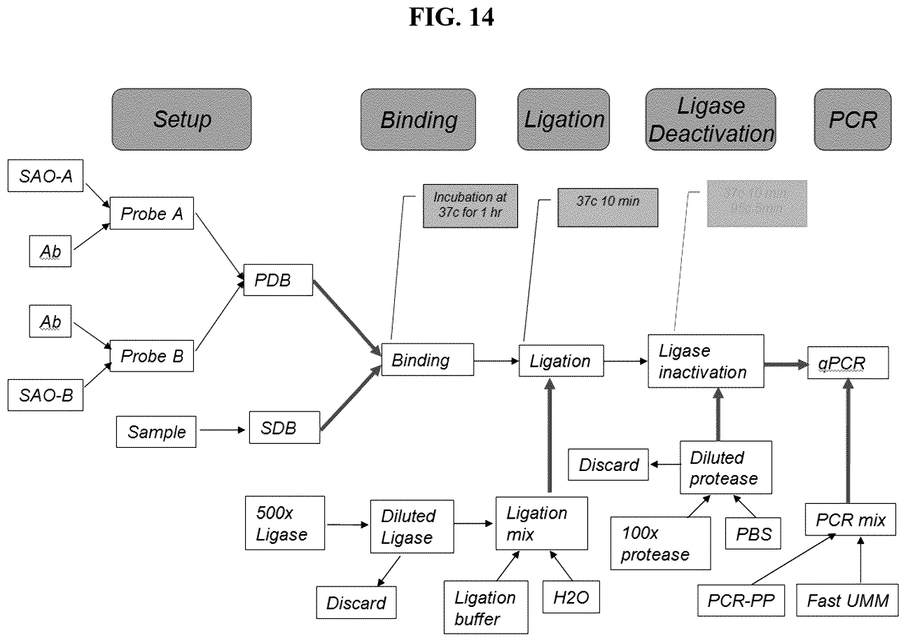

An example method 1400 for performing a PLA is shown in FIG. 14. Referring to FIG. 14, a binding reagent may be set up, or alternatively, provided. The binding reagent may be a probe mix containing at least two probes (Probe A and Probe B). The at least two probes may be primary antibodies (Ab), or a fragment thereof, each having a binding affinity or specificity to a ligand (e.g., a target protein analyte) in a sample (e.g., a biological sample). The at least two probes can have differing affinities or specificity to the target protein analyte, for example, binding to different epitopes of the target protein analyte. Each of the probes may also contain an oligonucleotide. The oligonucleotide of one probe may be coupled, optionally via intervening molecules, to the antibody probe via its 5′-end with its 3′-end being free; and the oligonucleotide of the other probe may be coupled, optionally via intervening molecules, to the probe via its 3′-end with its 5′-end being free. The oligonucleotides can be bound to the antibody probes, or antibody probe fragments, through one or more intervening molecules. The antibody probes, or antibody probe fragments, can be substituted with a DNA aptamer or other binding moiety.

In another example, the probe mix may contain at least two primary antibody probes and at least two secondary antibody probes (not shown) whereby the primary antibody probes are each conjugated to the respective oligonucleotide via one of the at least two secondary antibody probes to form the proximity probes. The proximity probes can have differing affinities or specificity to the target protein analyte, for example, binding to different epitopes of the target protein analyte.

Next, the probe mix may be brought into contact with the sample such that the at least two probes bind with specificity to the ligand in the sample to form proximity probes, which may cause the at least two probes to be in close proximity to another. The oligonucleotides that are attached to the probes may be brought into proximity to one another with the binding of the probes to the ligands. Then, the oligonucleotides are ligated to one another (e.g., through a ligation event) to form a ligated PLA product. The ligated PLA product may be a target nucleic acid comprising a nucleic acid template. The ligation event may be accomplished by adding ligation components, such as ligase (e.g., a small footprint ligase), adenosine triphosphate (ATP) and buffer-salt mixture, to the binding reaction.

In an example, a third oligonucleotide may be hybridized to both the oligonucleotides to form the target nucleic acid comprising the nucleic acid template. The third oligonucleotide may be referred to herein as a “splinter oligonucleotide” or “splinter oligo” in that it splints and joins the free ends of the oligonucleotides. The third oligonucleotide can be free or attached to a third proximity probe.

The ligase may then deactivated (e.g., by protease digestion) to prevent any further ligation of unbound oligonucleotides. The nucleic acid template of the target nucleic acid may then amplified by PCR to produce DNA amplicons. For instance, a real-time polymerase chain reaction (PCR) mixture (e.g., a mixture including a forward primer, a reverse primer, and a heat stable DNA polymerase) may be is added to a reaction mixture containing the nucleic acid template, and the quantity of the PCR product may be determined by quantitative PCR (qPCR) or real-time PCR. In an example, the forward primer may hybridize to a first strand of a double-stranded nucleic acid template, and the reverse primer may hybridize to a second strand of the double-stranded nucleic acid template.

An example of a PLA system may include, for example, TaqMan® Protein Assays. TaqMan® Protein Assays are an adapted form of PLA that combine antibody-protein binding with detection of the nucleic acid by real-time PCR or qPCR. In an example, formation of the target nucleic acid, and subsequent amplification of the nucleic acid template, can be combined in a single operation, for example, by using a mixture of reagents and allowing the reactions to occur in the same chamber.

As mentioned above, the contacting of the binding reagent with said analyte may include performing a proximity extension assay (PEA). In an embodiment, PEA may be performed using starting materials (e.g., analyte, capture agent, and binding reagent) and operations similar to those described in the PLA discussed above, except that the oligonucleotide coupled to one of the proximity probes is double-stranded with a 3′-overhang. Thus, in the presence of the target protein analyte, the oligonucleotides may hybridize to each other, which may result in extension of the oligonucleotide having the 3′-overhang to form a PLA product. The PLA product may be a target nucleic acid comprising a nucleic acid template. The nucleic acid template may then be amplified by real-time PCR or qPCR (as described above), and detected to determine the presence of the ligand within the sample.

In another example, PEA is performed using starting materials (e.g., analyte, capture agent, and binding reagent) and operations similar to those described in the PLA discussed above, except that the oligonucleotides are single-stranded oligonucleotides that hybridize directly to each other, resulting in extension of one of the oligonucleotides by DNA polymerase to form a PLA product. The PLA product may be a target nucleic acid comprising a nucleic acid template. The nucleic acid template may then be amplified by real-time PCR or qPCR (as described above) and detected to determine the presence of the ligand within the sample.

The nucleic acid template in the PLA or the PEA may be amplified by performing real-time PCR or qPCR using a microfluidic device as described below.

The method may comprise providing any of the microfluidic devices as described herein. The microfluidic device may comprise at least one channel. The channel may comprise an inlet, an outlet, or both an inlet and an outlet. In an example, the channel comprises a single inlet and does not include and outlet. In another example, the channel comprises an inlet and an outlet. The microfluidic device may further comprise a plurality of partitions (e.g., chambers) connected to the channel. The chambers may be connected to the channel by a plurality of siphon apertures. The microfluidic device may be sealed by a thin film (e.g., a thermoplastic thin film) disposed adjacent to a surface of the microfluidic device such that the thin film caps the channel, the plurality of chambers, the plurality of siphon apertures, or any combination thereof. Reagents, the nucleic acid molecules, or both may be applied to the inlet of the channel. The fluidic device may be filled by providing a first pressure differential between the reagent or nucleic acid molecules and the fluidic device, causing the reagent or nucleic acid molecules to flow into the fluidic device. The reagent or nucleic acid molecules may be partitioned into the chambers by applying a second pressure differential between the channel and the plurality of chambers to move the reagent or nucleic acid molecules into the plurality of chambers and to force gas within the plurality of chambers to pass through the thin film. Alternatively, or in addition to, the fluidic device may include a second channel configured to permit degassing or off-gassing. The second channel may be disposed adjacent to the plurality of chambers. The second pressure differential may be greater than the first pressure differential. A third pressure differential between the inlet and the outlet may be applied to introduce a fluid into the microchannel without introducing the fluid into the chambers. The third pressure differential may be less than the second pressure differential. A reagent may be added before, after, or at the same time as the nucleic acid molecules. A reagent may also be provided in one or more partitions of the device by another method. For example, a reagent may be deposited within one or more partitions prior to covering the one or more partitions with the thin film.

The inlet or the outlet, if present, of the device may be in fluid communication with a pneumatic pump or a vacuum system. The pneumatic pump or vacuum system may be a component of or separate from a system of the present disclosure. Filling and partitioning of a reagent or nucleic acid molecules may be performed by applying pressure differentials across various features of the fluidic device. Filling and partitioning of the reagent or nucleic acid molecules may be performed without the use of valves between the chambers and the channel to isolate reagent or nucleic acid molecules. For example, filling of the channel may be performed by applying a pressure differential between the reagent or nucleic acid molecules to be loaded and the channel. This pressure differential may be achieved by pressurizing the reagent or nucleic acid molecules or by applying vacuum to the channel. Filling the chambers may be performed by applying a pressure differential between the channel and the chambers. This may be achieved by pressurizing the channel or by applying a vacuum to the chambers. Partitioning the sample or reagent may be performed by applying a pressure differential between a fluid and the channel. This pressure differential may be achieved by pressurizing the fluid or by applying a vacuum to the channel.

The microfluidic device may include a thin film or second channel (e.g., off-gas channel) that may have different permeability characteristics under different applied pressure differentials. For example, the thin film or second channel may prevent gas flow at the first and third pressure differentials (e.g., low pressure), which may be smaller magnitude pressure differentials. The thin film or second channel may permit gas flow at the second pressure differential (e.g., high pressure), which may be a higher magnitude pressure differential. The first and third pressure differentials may be the same or they may be different. The first pressure differential may be the difference in pressure between the reagent in the inlet or outlet and the microfluidic device. During filling of the microfluidic device, the pressure of the reagent may be higher than the pressure of the microfluidic device. During filling of the fluidic device, the pressure difference between the reagent and the fluidic device (e.g., low pressure) may be less than or equal to about 8 pounds per square inch (psi), less than or equal to about 6 psi, less than or equal to about 4 psi, less than or equal to about 2 psi, less than or equal to about 1 psi, or less. In some examples, during filling of the fluidic device, the pressure differential between the reagent and the microfluidic device may be from about 1 psi to about 8 psi. In some examples, during filling of the fluidic device, the pressure differential between the reagent and the microfluidic device may be from about 1 psi to about 6 psi. In some examples, during filling of the microfluidic device, the pressure differential between the reagent and the microfluidic device may be from about 1 psi to about 4 psi. The fluidic device may be filled by applying a pressure differential between the reagent and the fluidic device for less than or equal to about 20 minutes, less than or equal to about 15 minutes, less than or equal to about 10 minutes, less than or equal to about 5 minutes, less than or equal to about 3 minutes, less than or equal to about 2 minutes, less than or equal about 1 minute, or less.

A filled microfluidic device may have nucleic acid molecules or one or more reagents in the channel, siphon apertures, chambers, or any combination thereof. Backfilling of the nucleic acid molecules or the one or more reagents into the chambers may occur upon filling of the fluidic device or may occur during application of a second pressure differential. The second pressure differential (e.g., high pressure) may correspond to the difference in pressure between the channel and the plurality of chambers. During application of the second pressure differential a first fluid (e.g., gas or liquid) in the higher pressure domain may push a second fluid (e.g., gas) in the lower pressure domain through the thin film and out of the fluidic device. The first and second fluids may comprise a liquid or a gas. The liquid may comprise an aqueous mixture or an oil mixture. The second pressure differential may be achieved by pressurizing the channel. Alternatively, or in addition, the second pressure differentially may be achieved by applying a vacuum to the chambers. During application of the second pressure differential, nucleic acid molecules or reagents in the channel may flow into the chambers. Additionally, during the application of the second pressure differential gas trapped within the siphon apertures, chambers, and channel may outgas through the thin film or through one or more walls of the chambers and into a second channel (e.g., off-gas channel). During backfilling and outgassing of the chambers, the pressure differential between the chambers and the channel may be greater than or equal to about 6 psi, greater than or equal to about 8 psi, greater than or equal to about 10 psi, greater than or equal to about 12 psi, greater than or equal to about 14 psi, greater than or equal to about 16 psi, greater than or equal to about 18 psi, greater than or equal to about 20 psi, or greater. In some examples, during backfilling of the chambers, the pressure differential between the chambers and the channel is from about 8 psi to about 20 psi. In some examples, during backfilling of the chambers, the pressure differential between the chambers and the channel is from about 8 psi to about 18 psi. In some examples, during backfilling of the chambers, the pressure differential between the chambers and the channel is from about 8 psi to about 16 psi. In some examples, during backfilling of the chambers, the pressure differential between the chambers and the microchannel is from about 8 psi to about 14 psi. In some examples, during backfilling of the chambers, the pressure differential between the chambers and the channel is from about 8 psi to about 12 psi. In some examples, during backfilling of the chambers, the pressure differential between the chambers and the channel is from about 8 psi to about 10 psi. The chambers may be backfilled and outgassed by applying a pressure differential for more than about 5 minutes, more than about 10 minutes, more than about 15 minutes, more than about 20 minutes, more than about 25 minutes, more than about 30 minutes, or more.

Partitioning of the nucleic acid molecules may be verified by the presence of an indicator within the reagent. An indicator may include a molecule comprising a detectable moiety. The detectable moiety may include radioactive species, fluorescent labels, chemiluminescent labels, enzymatic labels, colorimetric labels, or any combination thereof. Non-limiting examples of radioactive species include ³H, ¹⁴C, ²²Na, ³²P, ³³P, ³⁵S, ⁴²K, ⁴⁵Ca, ⁵⁹Fe, ¹²³I, ¹²⁴I, ¹²⁵I, ¹³¹I, or ²⁰³Hg. Non-limiting examples of fluorescent labels include fluorescent proteins, optically active dyes (e.g., a fluorescent dye), organometallic fluorophores, or any combination thereof. Non-limiting examples of chemiluminescent labels include enzymes of the luciferase class such as Cypridina, Gaussia, Renilla, and Firefly luciferases. Non-limiting examples of enzymatic labels include horseradish peroxidase (HRP), alkaline phosphatase (AP), beta galactosidase, glucose oxidase, or other labels.

An indicator molecule may be a fluorescent molecule. Fluorescent molecules may include fluorescent proteins, fluorescent dyes, and organometallic fluorophores. The indicator molecule may be a protein fluorophore. Protein fluorophores may include green fluorescent proteins (GFPs, fluorescent proteins that fluoresce in the green region of the spectrum, generally emitting light having a wavelength from 500-550 nanometers), cyan-fluorescent proteins (CFPs, fluorescent proteins that fluoresce in the cyan region of the spectrum, generally emitting light having a wavelength from 450-500 nanometers), red fluorescent proteins (RFPs, fluorescent proteins that fluoresce in the red region of the spectrum, generally emitting light having a wavelength from 600-650 nanometers). Non-limiting examples of protein fluorophores include mutants and spectral variants of AcGFP, AcGFP1, AmCyan, AmCyan1, AQ143, AsRed2, Azami Green, Azurite, BFP, Cerulean, CFP, CGFP, Citrine, copGFP, CyPet, dKeima-Tandem, DsRed, dsRed-Express, DsRed-Monomer, DsRed2, dTomato, dTomato-Tandem, EBFP, EBFP2, ECFP, EGFP, Emerald, EosFP, EYFP, GFP, HcRed-Tandem, HcRed1, JRed, Katuska, Kusabira Orange, Kusabira Orange2, mApple, mBanana, mCerulean, mCFP, mCherry, mCitrine, mECFP, mEmerald, mGrape1, mGrape2, mHoneydew, Midori-Ishi Cyan, mKeima, mKO, mOrange, mOrange2, mPlum, mRaspberry, mRFP1, mRuby, mStrawberry, mTagBFP, mTangerine, mTeal, mTomato, mTurquoise, mWasabi, PhiYFP, ReAsH, Sapphire, Superfolder GFP, T-Sapphire, TagCFP, TagGFP, TagRFP, TagRFP-T, TagYFP, tdTomato, Topaz, TurboGFP, Venus, YFP, YPet, ZsGreen, and ZsYellow1.

The indicator molecule may be a fluorescent dye. Non-limiting examples of fluorescent dyes include SYBR green; SYBR blue; DAPI; propidium iodine; Hoeste; SYBR gold; ethidium bromide; acridines; proflavine; acridine orange; acriflavine; fluorcoumanin; ellipticine; daunomycin; chloroquine; distamycin D; chromomycin; homidium; mithramycin; ruthenium polypyridyls; anthramycin; phenanthridines and acridines; propidium iodide; hexidium iodide; dihydroethidium; ethidium monoazide; ACMA; Hoechst 33258; Hoechst 33342; Hoechst 34580; DAPI; acridine orange; 7-AAD; actinomycin D; LDS751; hydroxystilbamidine; SYTOX Blue; SYTOX Green; SYTOX Orange; POPO-1; POPO-3; YOYO-1; YOYO-3; TOTO-1; TOTO-3; JOJO-1; LOLO-1; BOBO-1; BOBO-3; PO-PRO-1; PO-PRO-3; BO-PRO-1; BO-PRO-3; TO-PRO-1; TO-PRO-3; TO-PRO-5; JO-PRO-1; LO-PRO-1; YO-PRO-1; YO-PRO-3; PicoGreen; OliGreen; RiboGreen; SYBR Gold; SYBR Green I; SYBR Green II; SYBR DX; SYTO-40, SYTO-41, SYTO-42, SYTO-43, SYTO-44, and SYTO-45 (blue); SYTO-13, SYTO-16, SYTO-24, SYTO-21, SYTO-23, SYTO-12, SYTO-11, SYTO-20, SYTO-22, SYTO-15, SYTO-14, and SYTO-25 (green); SYTO-81, SYTO-80, SYTO-82, SYTO-83, SYTO-84, and SYTO-85 (orange); SYTO-64, SYTO-17, SYTO-59, SYTO-61, SYTO-62, SYTO-60, and SYTO-63 (red); fluorescein; fluorescein isothiocyanate (FITC); tetramethyl rhodamine isothiocyanate (TRITC); rhodamine; tetramethyl rhodamine; R-phycoerythrin; Cy-2; Cy-3; Cy-3.5; Cy-5; Cy5.5; Cy-7; Texas Red; Phar-Red; allophycocyanin (APC); Sybr Green I; Sybr Green II; Sybr Gold; CellTracker Green; 7-AAD; ethidium homodimer I; ethidium homodimer II; ethidium homodimer III; umbelliferone; eosin; green fluorescent protein; erythrosin; coumarin; methyl coumarin; pyrene; malachite green; stilbene; lucifer yellow; cascade blue; dichlorotriazinylamine fluorescein; dansyl chloride; fluorescent lanthanide complexes such as those including europium and terbium; carboxy tetrachloro fluorescein; 5 or 6-carboxy fluorescein (FAM); 5- (or 6-) iodoacetamidofluorescein; 5-{[2(and 3)-5-(Acetylmercapto)-succinyl]amino} fluorescein (SAMSA-fluorescein); lissamine rhodamine B sulfonyl chloride; 5 or 6 carboxy rhodamine (ROX); 7-amino-methyl-coumarin; 7-Amino-4-methylcoumarin-3-acetic acid (AMCA); BODIPY fluorophores; 8-methoxypyrene-1;3;6-trisulfonic acid trisodium salt; 3;6-Disulfonate-4-amino-naphthalimide; phycobiliproteins; AlexaFluor 350, 405, 430, 488, 532, 546, 555, 568, 594, 610, 633, 635, 647, 660, 680, 700, 750, and 790 dyes; DyLight 350, 405, 488, 550, 594, 633, 650, 680, 755, and 800 dyes; and other fluorophores.

The indicator molecule may be an organometallic fluorophore. Non-limiting examples of organometallic fluorophores include lanthanide ion chelates, non-limiting examples of which include tris(dibenzoylmethane) mono(1,10-phenanthroline)europium(lll), tris(dibenzoylmethane) mono(5-amino-1,10-phenanthroline)europium (lll), and Lumi4-Tb cryptate.

The method may further comprise amplifying the nucleic acid molecule. Amplification of the nucleic acid molecule may increase a concentration of the nucleic acid molecule and, thus, increase detectable signal associated with the nucleic acid molecule. Prior to amplification, the nucleic acid molecule may be cleaved or otherwise removed from the binding probe.

Alternatively, or in addition to, the nucleic acid may be amplified while coupled to the binding probe. The nucleic acid molecule may be a single stranded nucleic acid molecule or may be a double stranded nucleic acid molecule. In an example, the nucleic acid molecule is a double stranded nucleic acid molecule and is denatured prior to amplification. The nucleic acid molecule may have a consensus sequence complementary to a universal primer. The universal primer may be extended to amplify the nucleic acid molecule. Alternatively, or in addition to, the nucleic acid molecule may have a consensus sequence for a primer specific to the nucleic acid molecule.

The microfluidic device may be filled with one or more amplification reagents such as nucleic acid molecules, components for an amplification reaction (e.g., primers, polymerases, and deoxyribonucleotides), an indicator molecule, and an amplification probe. Amplification reactions may involve thermal cycling the plurality of microchambers or a subset thereof, as described herein. Detection of nucleic acid amplification may be performed by collecting signals from (e.g., imaging) the plurality of chambers of the microfluidic device or a subset thereof. Nucleic acid molecules may be quantified by counting the microchambers in which the nucleic acid molecules are successfully amplified and applying Poisson statistics. The nucleic acid molecule may be partitioned such that a partition comprises one or less nucleic acid molecule. Alternatively, or in addition to, a partition may include multiple nucleic acid molecules. Nucleic acid molecules may also be quantified by processing signals collected at different time points throughout an amplification reaction. For example, one or more signals may be collected during each thermal cycle (e.g., each amplification cycle) of a nucleic acid amplification reaction and the signals can be used to determine an amplification rate as in, e.g., real-time or quantitative polymerase chain reaction (real-time PCR or qPCR). Nucleic acid amplification and quantification may be performed in a single integrated unit, e.g., within a given partition or a subset of the plurality of partitions of the device.

A variety of nucleic acid amplification reactions may be used to amplify the nucleic acid molecule in a sample to generate an amplified product. Amplification of a nucleic acid target may be linear, exponential, or a combination thereof. Non-limiting examples of nucleic acid amplification methods include primer extension, polymerase chain reaction, reverse transcription, isothermal amplification, ligase chain reaction, helicase-dependent amplification, asymmetric amplification, rolling circle amplification, and multiple displacement amplification. The amplification product of an amplification reaction may be DNA or RNA. For samples including DNA molecules, any DNA amplification method may be employed. DNA amplification methods include, but are not limited to, PCR, real-time PCR, assembly PCR, asymmetric PCR, digital PCR, dial-out PCR, helicase-dependent PCR, nested PCR, hot start PCR, inverse PCR, methylation-specific PCR, miniprimer PCR, multiplex PCR, overlap-extension PCR, thermal asymmetric interlaced PCR, touchdown PCR, and ligase chain reaction. DNA amplification may be linear, exponential, or any combination thereof. DNA amplification may also be achieved with digital PCR (dPCR), real-time quantitative PCR (qPCR), or quantitative digital PCR (qdPCR), as described herein.

Reagents used for nucleic acid amplification may include polymerizing enzymes, reverse primers, forward primers, and amplification probes. Examples of polymerizing enzymes include, without limitation, nucleic acid polymerase, transcriptase, or ligase (i.e., enzymes which catalyze the formation of a bond). The polymerizing enzyme can be naturally occurring or synthesized. Examples of polymerases include a DNA polymerase, and RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase Pwo polymerase, VENT polymerase, DEEPVENT polymerase, Ex-Taq polymerase, LA-Taw polymerase, Sso polymerase Poc polymerase, Pab polymerase, Mth polymerase ES4 polymerase, Tru polymerase, Tac polymerase, Tne polymerase, Tma polymerase, Tca polymerase, Tih polymerase, Tfi polymerase, Platinum Taq polymerases, Tbr polymerase, Tfl polymerase, Pfutubo polymerase, Pyrobest polymerase, KOD polymerase, Bst polymerase, Sac polymerase, Klenow fragment polymerase with 3′ to 5′ exonuclease activity, and variants, modified products and derivatives thereof. For a Hot Start polymerase, denaturation at a temperature from about 92° C. to 95° C. for a time period from about 2 minutes to 10 minutes may be used.

A nucleic acid amplification reaction may involve an amplification probe. An amplification probe may be a sequence-specific oligonucleotide probe. The amplification probe may be optically active when hybridized with an amplification product. The amplification probe may be detectable as nucleic acid amplification progresses. The intensity of a signal collected from a plurality of partitions including nucleic acid molecules (e.g., optical signal) may be proportional to the amount of amplified product included in the partitions. For example, the signal collected from a particular partition may be proportional to the amount of amplified product in that particular partition. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, Lion probes, locked nucleic acid probes, or molecular beacons. Non-limiting examples of quenchers that may be useful in blocking the optical activity of the probe include Black Hole Quenchers (BHQ), Iowa Black FQ and RQ quenchers, or Internal ZEN Quenchers. Alternatively, or in addition, the probe or quencher may be any probe that is useful in the context of the methods of the present disclosure.

The amplification probe may be a dual labeled fluorescent probe. The dual labeled probe may include a fluorescent reporter and a fluorescent quencher linked with a nucleic acid. The fluorescent reporter and fluorescent quencher may be positioned in close proximity to each other. The close proximity of the fluorescent reporter and fluorescent quencher may block the optical activity of the fluorescent reporter. The dual labeled probe may bind to the nucleic acid molecule to be amplified. During amplification, the fluorescent reporter and fluorescent quencher may be cleaved by the exonuclease activity of the polymerase. Cleaving the fluorescent reporter and quencher from the amplification probe may cause the fluorescent reporter to regain its optical activity and enable detection. The dual labeled fluorescent probe may include a 5′ fluorescent reporter with an excitation wavelength maximum of about 450 nanometers (nm), 500 nm, 525 nm, 550 nm, 575 nm, 600 nm, 625 nm, 650 nm, 675 nm, 700 nm, or higher and an emission wavelength maximum of about 500 nm, 525 nm, 550 nm, 575 nm, 600 nm, 625 nm, 650 nm, 675 nm, 700 nm, or higher. The dual labeled fluorescent probe may also include a 3′ fluorescent quencher. The fluorescent quencher may quench fluorescent emission wavelengths between about 380 nm and 550 nm, 390 nm and 625 nm, 470 nm and 560 nm, 480 nm and 580 nm, 550 nm and 650 nm, 550 nm and 750 nm, or 620 nm and 730 nm.

Nucleic acid amplification may include multiple cycles of thermal cycling (e.g., multiple amplification cycles). Any suitable number of cycles may be performed. The number of cycles performed may be more than about 5, more than about 10, more than about 15, more than about 20, more than about 30, more than about 40, more than about 50, more than about 60, more than about 70, more than about 80, more than about 90, more than about 100 cycles, or more. The number of cycles performed may depend upon the number of cycles to obtain detectable amplification products. For example, the number of cycles to detect nucleic acid amplification during PCR (e.g., dPCR, qPCR, or qdPCR) may be less than or equal to about 100, less than or equal to about 90, less than or equal to about 80, less than or equal to about 70, less than or equal to about 60, less than or equal to about 50, less than or equal to about 40, less than or equal to about 30, less than or equal to about 20, less than or equal to about 15, less than or equal to about 10, less than or equal to about 5 cycles, or less.

The time to reach a detectable amount of amplification product may vary depending upon, for example, the particular nucleic acid molecules, the reagents used, the amplification reaction used, the number of amplification cycles used, and the reaction conditions. The time to reach a detectable amount of amplification product may be about 120 minutes or less, 90 minutes or less, 60 minutes or less, 50 minutes or less, 40 minutes or less, 30 minutes or less, 20 minutes or less, 10 minutes or less, or 5 minutes or less.

The method may further comprise denaturing the nucleic acid molecule or derivative thereof. In an example, the method further comprises denaturing amplification products of the nucleic acid molecule. The nucleic acid molecule or derivative thereof may be denatured using thermal energy, acids or bases (e.g., sodium hydroxide treatment), organic solvents, salts, or any combination thereof. Denaturation of the nucleic acid molecule may be reversible (e.g., via thermal denaturation) or irreversible (e.g., via salts or organic solvents). In an example, the nucleic acid is denatured by thermal energy. Denaturation temperatures may vary depending upon, for example, the nucleic acid molecule, the reagents used, and the reaction conditions.

The nucleic acid molecule or derivative thereof may be thermally denatured via controlled heating of the nucleic acid molecule or derivative thereof. In an example, the nucleic acid molecule(s) are disposed in a plurality of partitions and the partitions undergo controlled heating. Controlled heating may include resistive heating, radiative heating, conductive heating, convective heating, or any combination thereof. Thermal denaturation may include controlled heating of the nucleic acid molecule or derivative thereof to a temperature of from about 60° C. to 70° C., 60° C. to 80° C., 60° C. to 90° C., 60° C. to 100° C., 60° C. to 110° C., 70° C. to 80° C., 70° C. to 90° C., 70° C. to 100° C., 70° C. to 110° C., 80° C. to 90° C., 80° C. to 100° C., 80° C. to 110° C., 90° C. to 100° C., 90° C. to 110° C., or 100° C. to 110° C. for a given period of time. In an example, the nucleic acid molecule may undergo controlled heating from about 60° C. to about 90° C. for a given time. In another example, the nucleic acid molecule may undergo controlled heating from about 65° C. to about 85° C. for a given time. Thermal denaturation may include controlled heating of the nucleic acid molecule or derivative thereof to a temperature of greater than or equal to about 60° C., 65° C., 70° C., 80° C., 85° C., 90° C., 95° C., or higher for a given period of time.

The duration of thermal denaturation may vary depending upon, for example, the particular nucleic acid molecule, the reagents used, and the reaction conditions. The duration of thermal denaturation may be less than or equal to about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. Alternatively, the duration for denaturation may be no more than about 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Controlled heating of a subset of the plurality of partitions of a device may be performed at any useful rate and over any useful temperature range. For example, controlled heating may be performed from a lower temperature of at least about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C., or more. Controlled heating may be performed to an upper temperature of at least about 35° C., about 40° C., about 45° C., about 50° C., about 55° C., about 60° C., about 65° C., about 70° C., about 75° C., about 80° C., about 85° C., about 90° C., about 91° C., about 92° C., about 93° C., about 94° C., about 95° C., about 96° C., about 97° C., about 98° C., about 99° C., or about 100° C., or more. Temperature may be increased by any useful increment. For example, temperature may be increased by at least about 0.01° C., about 0.05° C., about 0.1° C., about 0.2° C., about 0.3° C., about 0.4° C., about 0.5° C., about 1° C., about 2° C., about 3° C., about 4° C., about 5° C., or about 10° C., or more. Controlled heating may also occur over evenly or evenly spaced temperature increments. For example, temperature may be increased by about 0.1° C. over a range where significant melting of a nucleic acid molecule is expected (e.g., fine grain measurement) and by about 1° C. over a range where no significant melting of a nucleic acid molecule is expected (e.g., coarse grain measurement). Controlled heating may be performed at any useful rate such as at least about 0.0001° C./second, about 0.0002° C./second, about 0.0003° C./second, about 0.0004° C./second, about 0.0005° C./second, about 0.0006° C./second, about 0.0007° C./second, about 0.0008° C./second, about 0.0009° C./second, about 0.001° C./second, about 0.002° C./second, about 0.003° C./second, about 0.004° C./second, about 0.005° C./second, about 0.006° C./second, about 0.007° C./second, about 0.008° C./second, about 0.009° C./second, about 0.01° C./second, about 0.02° C./second, about 0.03° C./second, about 0.04° C./second, about 0.05° C./second, about 0.06° C./second, about 0.07° C./second, about 0.08° C./second, about 0.09° C./second, about 0.1° C./second, about 0.2° C./second, about 0.3° C./second, about 0.4° C./second, about 0.5° C./second, about 0.6° C./second, about 0.7° C./second, about 0.8° C./second, about 0.9° C./second, about 1° C./second, about 2° C./second, about 3° C./second, about 4° C./second, and about 5° C./second, or more. A thermal unit (e.g., a heater) carrying out the controlled heating process may maintain a given temperature for any useful duration. For example, a given temperature may be maintained for at least about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 25 seconds, about 30 seconds, about 45 seconds, about 60 seconds, about 70 seconds, about 80 seconds, about 90 seconds, about 100 seconds, about 110 seconds, about 120 seconds, about 130 seconds, about 140 seconds, about 150 seconds, about 160 seconds, about 170 seconds, about 180 seconds, about 190 seconds, about 200 seconds, about 210 seconds, about 220 seconds, about 230 seconds, about 240 seconds, about 250 seconds or about 300 seconds, or more.

The method may further include collecting signals from the nucleic acid molecule or derivative thereof (e.g., amplification products of the nucleic acid molecule) during denaturation of the nucleic acid molecule. Signals may include optical signals, electrical signals, or any combination thereof. In an example, signals collected are optical signals. Collection of optical signals may include imaging partitions of the microfluidic device or a portion of the partitions of the microfluidic device. Signals may be collected from the subset of the plurality of partitions at any selected time points. For example, signal may be collected at least about every 1 second, about every 2 seconds, about every 3 seconds, about every 4 seconds, about every 5 seconds, about every 6 seconds, about every 7 seconds, about every 8 seconds, about every 9 seconds, about every 10 seconds, about every 20 seconds, about every 30 seconds, about every 45 seconds, about every 60 seconds, about every 70 seconds, about every 80 seconds, about every 90 seconds, about every 100 seconds, about every 110 seconds, about every 120 seconds, about every 130 seconds, about every 140 seconds, about every 150 seconds, about every 160 seconds, about every 170 seconds, about every 180 seconds, about every 190 seconds, about every 200 seconds, about every 210 seconds, about every 220 seconds, about every 230 seconds, about every 240 seconds, about every 250 seconds, or about every 300 seconds, or more. Alternatively, or in addition to, signals may be collected at select temperature intervals. For example, signals may be collected at temperature intervals of less than or equal to about 5° C., 4° C., 3° C., 2.5° C., 2° C., 1.5° C., 1° C., 0.5° C., 0.25° C., or less. Signals may be collected (e.g., images taken) from the microfluidic device or a subset of the plurality of partitions (e.g., microchambers) thereof. Collecting signals may comprise taking images of the device or a subset of the plurality of partitions thereof. Signals (e.g., images) may be collected from single microchambers, an array of microchambers, or of multiple arrays of microchambers concurrently. Signals may be collected through the body of the microfluidic device, through the thin film of the microfluidic device, or both. The body of the microfluidic device may be substantially optically transparent. Alternatively, the body of the microfluidic device may be substantially optically opaque. Similarly, the thin film may be substantially optically transparent. Alternatively, the body of the microfluidic device may be substantially optically opaque

The method may further include using intercalating dye. The intercalating dyes may generate the detectable signals. The detectable signals produced by the method may be presented or read as raw fluorescence units, relative fluorescence units, copy numbers (cp), copy numbers in relation to volume (e.g., cp/μL), critical threshold of amplification (Ct), amplification cycles, concentration, absolute numbers, derivative reporter, arbitrary units, or any combination thereof. Intercalating dyes may be inserted into the nucleic acid molecule during amplification of the nucleic acid molecule. Intercalating dyes may non-specifically interact or bind to double stranded nucleic acid molecules. Association of the intercalating dye with a double stranded nucleic acid molecule may permit detectable fluorescence of the intercalating dye. Denaturation of the nucleic acid molecule may quench or otherwise extinguish the fluorescence signal from the intercalating dye. Non-limiting examples of intercalating dyes include SYBR green, EvaGreen, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines, LCGReen, or any combination thereof.

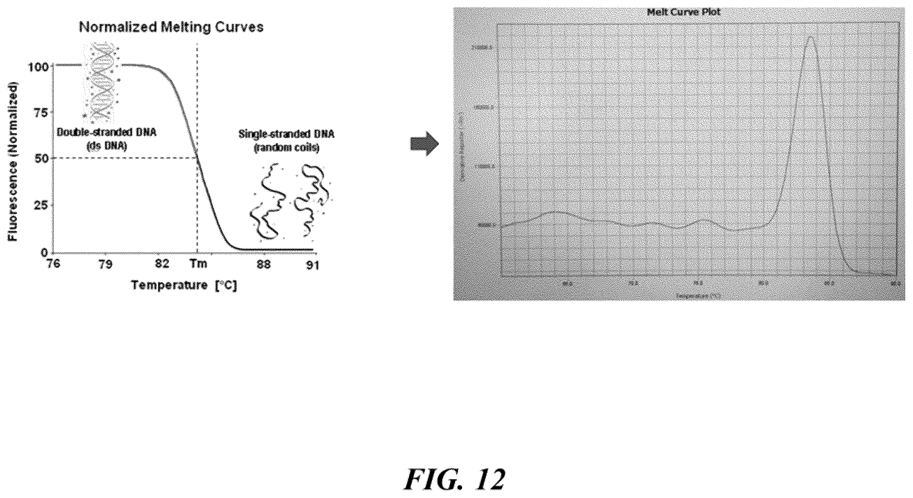

The method may further include processing the collected signals to generate denaturation profiles of the nucleic acid molecules. Alternatively, or in addition to, the collected signals may be processed to generate denaturation profiles for an individual partition comprising one or more nucleic acid molecules. Processing collected signals may comprise using the signals to generate denaturation curves, such as signal versus temperature curves or signal versus concentration curves (e.g., for base, salt, or organic solvent denaturation). Signal may include optical signal (e.g., fluorescence intensity) or non-optical signals (e.g., electrical signals). Processing the signals may further include plotting the negative first derivative of the denaturation curve to determine the denaturation temperature or denaturant concentration. Processing the signals may further include performing melt curve analysis to determine a denaturation profile or dissociation characteristics (e.g., melting temperature) of the nucleic acid molecule or plurality of nucleic acid molecules. FIG. 12 illustrates an example digital melt analysis using an intercalating dye and detection processing to generate the denaturation profile. The intercalating dye may generate a signal when associated with a double stranded nucleic acid molecule. As the double stranded nucleic acid molecule is denatured and forms random coils, the intercalating dye may disassociate from the single stranded nucleic acid molecules and the signal may decrease or go to zero.

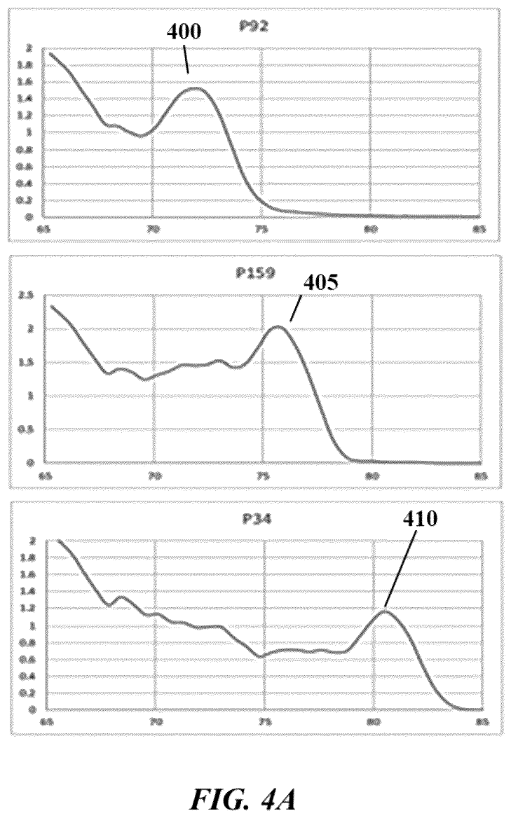

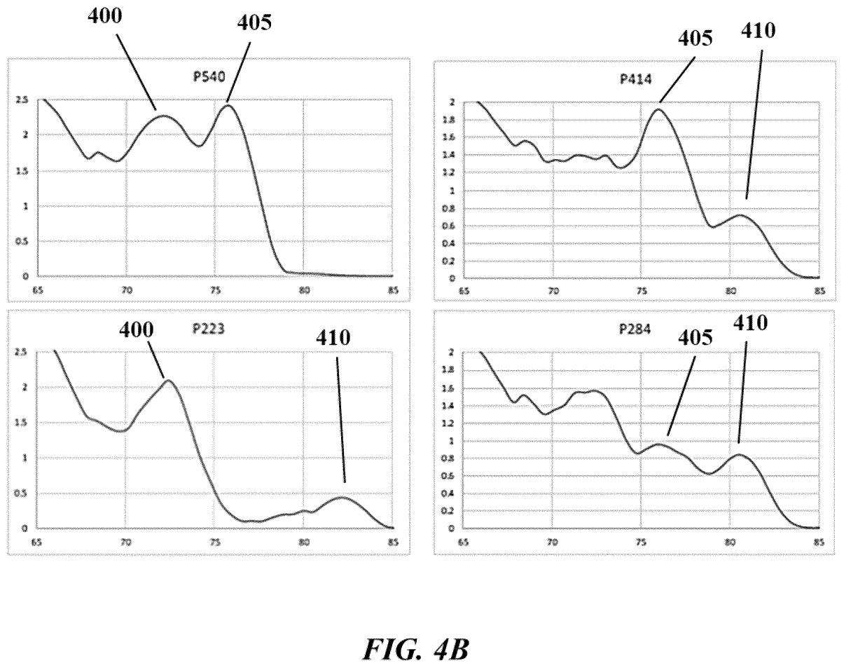

FIG. 4A-FIG. 4B illustrate example denaturation profiles for nucleic acid molecules. FIG. 4A illustrates example denaturation profiles for three different example nucleic acid molecules. The three different nucleic acid molecules exhibit different melting points 400, 405, and 410, a type of denaturation profile. The three different melting temperatures of the three different nucleic acid molecules may permit identification of three different analytes. For example, a binding reagent with a first nucleic acid molecule may exhibit a first melting point 400. A second binding reagent with a second nucleic acid molecule may exhibit a second melting point 405 and a third binding reagent with a third nucleic acid molecule may exhibit a third melting point 410. The different melting points of the nucleic acid molecules may permit identification of the analytes that the three binding reagents have specificity for. The nucleic acid molecules may be partitioned such that a single nucleic acid molecule, or amplification product of the nucleic acid molecule, is present in a single partition. Alternatively, or in addition to, a partition may include more than one nucleic acid molecules, or amplification products thereof. FIG. 4B illustrates example denaturation profiles for example partitions including multiple nucleic acid molecules. The melting points 400, 405, and 410 may permit the nucleic acid molecules to be distinguished from one another such that the presence of the different nucleic acid molecules, and therefore the analytes associated with the nucleic acid molecules, may be detected and identified.

A nucleic acid molecule may have a different denaturation profile from another nucleic acid molecule. For example, a first nucleic acid molecule may generate a first denaturation profile and a second nucleic acid molecule may generate a second denaturation profile. The first denaturation profile may permit detection, identification, or quantification of a first analyte and the second denaturation profile may permit detection, identification, or quantification of a second analyte. In some cases, processing the collected signals may include generating 1, 2, 3, 4, 5, 6, 7, 8, 9, 10, 11, 12, or 13 denaturation profiles for 1, 2, 3, 4, 5, 6, 7, 8, 9, 10, 11, 12, or 13 nucleic acids that correspond to 1, 2, 3, 4, 5,6 7, 8, 9, 10, 11, 12, or 13 different analytes. In some cases, the nucleic acids and analytes are in a single sample or multiple samples. A denaturation profile of a nucleic acid molecule may be dependent upon nucleobases that make up the nucleic acid molecule, length (e.g., number of bases) of the nucleic acid molecule, type of nucleotides that make up the nucleic acid molecule (e.g., PNA, DNA, BNA, LNA, etc.), concentration of nucleic acid molecules, or any combination thereof. A nucleic acid molecule may be different from another nucleic acid molecule. A difference between the nucleic acid molecule and another nucleic acid molecule may permit the nucleic acid molecule to be distinguished via a denaturation provide from another nucleic acid molecule. A characteristic (e.g., melting temperature, concentration of denaturation, etc.) of the denaturation profile of the nucleic acid molecule may be at least about 0.5%, 1%, 2%, 3%, 4%, 5%, 6%, 8%, 10%, 12%, 15%, 20%, 30%, 40%, or more different from a denaturation profile of another nucleic acid molecule. For example, a melting temperature of the nucleic acid molecule may be at least about 0.5% different from a melting temperature of another nucleic acid molecule. In another example, a melting temperature of the nucleic acid molecule may be at least about 1% different from a melting temperature of another nucleic acid molecule. In another example, a melting temperature of the nucleic acid molecule may be at least about 2% different from a melting temperature of another nucleic acid molecule. In another example, a melting temperature of the nucleic acid molecule may be at least about 5% different from a melting temperature of another nucleic acid molecule.

In an example, a denaturation profile of a nucleic acid molecule includes a melting point of the nucleic acid molecule and is different from a denaturation profile (e.g., melting point) of another nucleic acid molecule. The melting point of a nucleic acid molecule may be at least about 0.1° C., 0.2° C., 0.3° C., 0.4° C., 0.5° C., 0.6° C., 0.8° C., 1° C., 1.5° C., 2° C., 3° C., 4° C., 5° C., 6° C., 8° C., 10° C., or more degrees different than a melting point of another nucleic acid molecule. In an example, the melting point of a nucleic acid molecule may be at least 0.25° C. different than a melting point of another nucleic acid molecule. In another example, the melting point of a nucleic acid may be at least 0.5° C. different than a melting point of another nucleic acid molecule. The melting point may be derived from a first derivative plot of the nucleic acid molecule. Alternatively, or in addition, melting curve subtraction may be used to distinguish a melting point of a nucleic acid molecule from another nucleic acid molecule and, thus, distinguish identity of one analyte from another.

Processing the signals may permit identification of the nucleic acid molecule, and therefore, the analytes associated with the nucleic acid molecules. The denaturation profile (e.g., or melting temperature) of the nucleic acid molecule may be compared to or matched with a denaturation profile from a database of denaturation profiles. The database of denaturation profiles may provide information regarding the nucleic acid molecule, binding reagent coupled to the nucleic acid molecule, specificity of the binding probe, or any combination thereof. This information may permit identification of the analyte (e.g., protein) for which the binding reagent couples to.

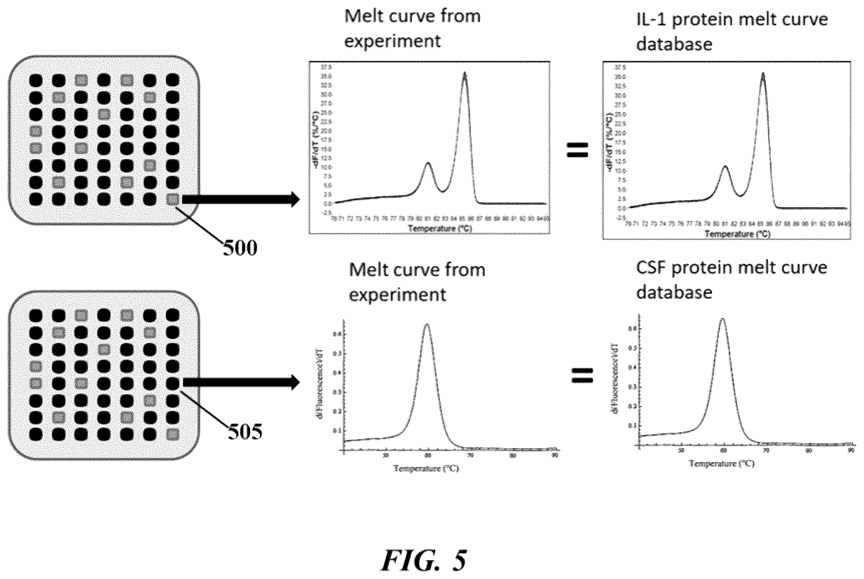

Additionally, or alternatively, processing may permit quantification of the analyte or analytes. The nucleic acid molecules may be partitioned into a plurality of partitions. Signals from the partitions may be collected and processed. A partition may be assigned as comprising no nucleic acid molecules or as comprising one or more nucleic acid molecules. For example, a partition may include a first and a second nucleic acid molecule. A number of partitions including the first nucleic acid molecule may be counted and permit quantification of the num nucleic acid molecules and, therefore, quantification of an analyte associated with the first nucleic acid molecule. A number of partitions including the second nucleic acid molecule may be counted and permit quantification of the number of second nucleic acid molecules and, therefore, quantification of an analyte associated with the second nucleic acid molecule. FIG. 5 schematically illustrates identification and quantification of example analytes. In this example, a sample comprising multiple analytes may be contacted with multiple binding reagents. First binding reagents with a binding probe specific for a first analyte may bind to the first analyte. Second binding reagents with a binding probe specific for a second analyte may bind to the second analyte. The first binding reagents and second binding reagents may first and second nucleic acid molecules, respectively. The first and second nucleic acid molecules may be partitioned and subjected to conditions to denature the first and second nucleic acid molecules. The first and second nucleic acid molecules may denature under different conditions such that the first and second nucleic acid molecules generate distinguishable denaturation profiles. During denaturation, signals associated with denaturation may be collected from the partitions to generate denaturation profiles for the individual partitions. A first partition 500 may include both the first and second nucleic acid molecule and may, therefore, generate a denaturation profile including denaturation signals from both the first and second nucleic acid molecules. A second partition 505 may include the first nucleic acid molecule and may generate a denaturation profile including denaturation signals from the first nucleic acid molecule. The denaturation profiles for the first and second nucleic acid molecules may be compared to a database of denaturation profiles to identify the analytes associated with the first and second nucleic acid molecules. The number of partitions comprising the first nucleic acid molecule, second nucleic acid molecule, or both the first and second nucleic acid molecule may be counted and processed to quantify the analytes associated with the first and second nucleic acid molecules.

In an example, the methods and systems provided herein may include using nucleic acid molecules that are produced from a proximity ligation assay (PLA).

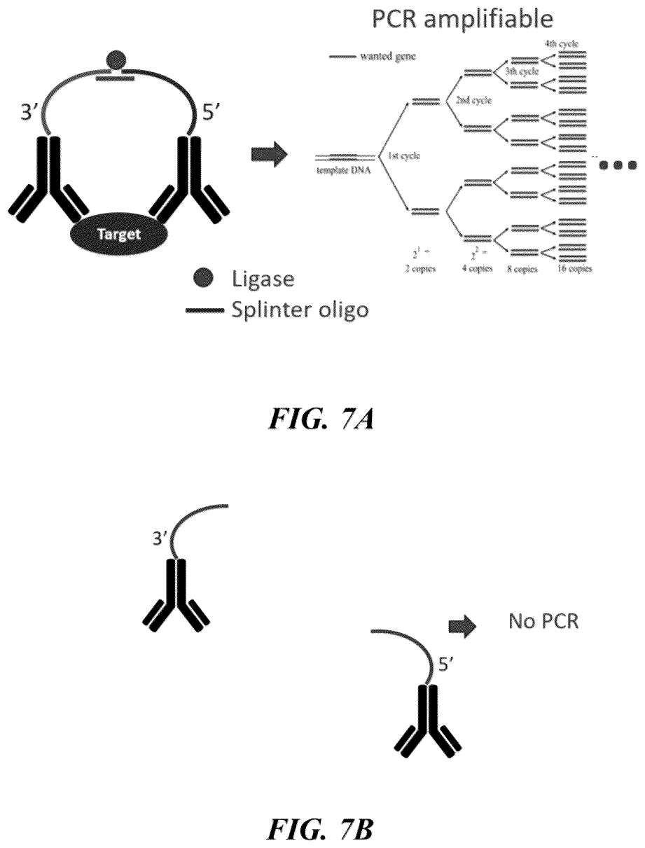

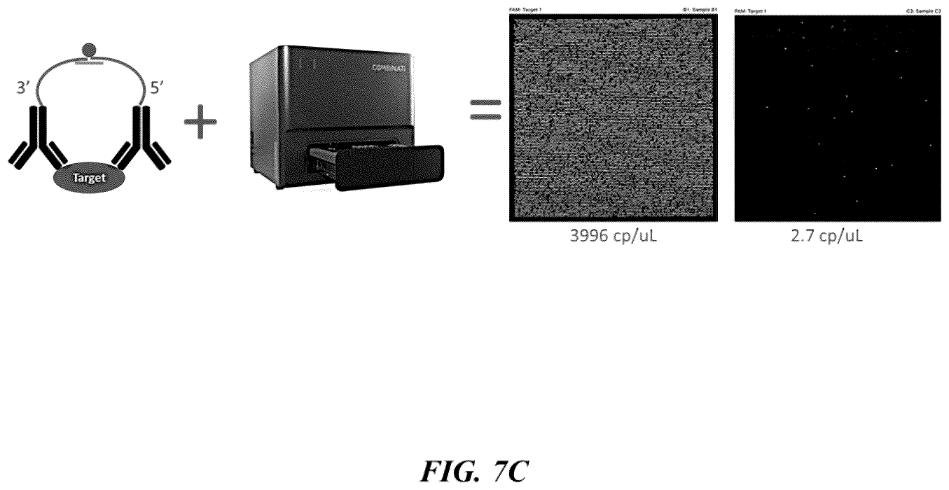

In such methods and systems, as illustrated, for example, in FIG. 7A the nucleic acid may be provided through a ligation reaction between two single stranded nucleic acid molecules conjugated to a binding reagent (e.g. antibodies or fragments thereof). In an example, the ligation reaction occurs if two binding agents bind to the same analyte, or sufficiently proximate targets, to thereby allow hybridization between both of the single stranded nucleic acids and a splinter oligo that spans a region of both of the single stranded nucleic acids. The splinter oligo may permit ligation of the single stranded nucleic acid molecules by bringing the 5′ and 3′ ends of the two nucleic acids together. In an example, if the two binding reagents are unable to bind the same analyte, or sufficiently proximate target analytes, then no ligation reaction occurs, as illustrated in FIG. 7B. PLA may have several advantages including few or no washing, small amounts of materials or labeling beads as compared to other analyte detection approaches, or lower cost imaging and automation equipment than other detection approaches. In an example, unbound binding reagents do not permit ligation of the single stranded nucleic acid molecules and, as such, no wash may be used. Furthermore, the PLA process may permit for more sensitive detection and an easier, faster, and less expensive workflow in combination with the digital PCR systems and methods described herein, as shown, for example, in FIG. 7C.

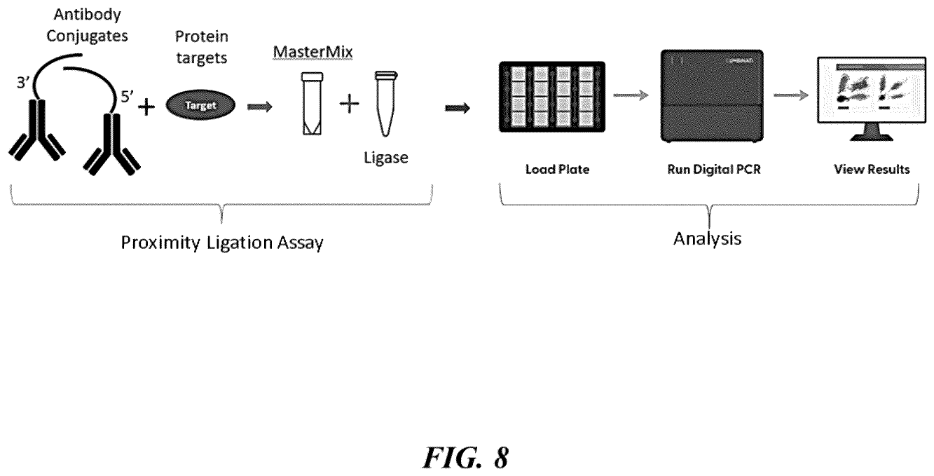

In an example, PLA may be integrated into a multiwell plate digital PCR (dPCR) workflow, as illustrated in FIG. 8. In some cases, a sample is provided that contains an analyte, such as a protein target of interest. In other cases, multiple samples are provided that contain multiple analytes. Sample may be mixed with antibody-ssDNA conjugates, splinter oligos, a master mix of PLA, dPCR buffers, a ligase to trigger the proximity ligase reaction, or any combination thereof. The reaction mixture(s) may then be loaded into a multiwell plate for dPCR, which may amplify the ligation reaction product nucleic acid as described herein for analysis (e.g., detection or quantification).

In some aspects, the PLA workflow may be integrated with a digital melt curve analysis as described herein. The ligated PLA product may serve as the nucleic acid for downstream amplification by dPCR or qPCR and subsequent denaturation profile analysis used to determine the concentration or quantification of an analyte or multiple analytes.

In some cases, the ligated PLA product may be generated by a recombinant or synthetic ligase. In some cases, the ligated PLA product may be generated by a ligase from a human, bacterial, viral, mammalian, eukaryotic, prokaryotic, archaea, or plant. In some cases, the ligase may be a fragment of a wild-type ligase or a fusion protein conjugated to another functional enzymatic domain. In some examples, the ligase may be provided in a master mix along with other buffers or PCR reagents. In some examples, the ligase may be provided in a plate or sample vessel prior to the addition of reagents or samples. In some examples, the ligase is added to the sample or binding reagent prior to addition to the reaction plate or vessel. In some examples, the nucleic acid for amplification is generated by contacting the ligase with a nucleic acid of one binding reagent and the nucleic acid of a second binding reagent. In some examples, the ligase is contacted with a first nucleic acid, a second nucleic acid, and a splinter oligo.

The splinter oligo (or splinter oligonucleotide) may be a polymeric form of nucleotides of any length. For example, the splinter oligo may include at least 1, 2, 3, 4, 5, 6, 8, 10, 15, 20, 30, 40, 50, 100, 500, 1000, or more nucleotides. The nucleotides may include deoxyribonucleotides, ribonucleotides, or analogs thereof. The splinter oligo may comprise deoxyribonucleic acid (DNA), ribonucleic acid (RNA), peptide nucleic acid (PNA), lock nucleic acid (LNA), bridge nucleic acid (BNA), or any combination thereof. A splinter oligo may include one or more subunits selected from adenosine (A), cytosine (C), guanine (G), thymine (T), and uracil (U), or variants thereof. A nucleotide can include A, C, G, T, or U, or variants thereof. A nucleotide can include any subunit that can be incorporated into a growing nucleic acid strand. Such subunit can be A, C, G, T, or U, or any other subunit that is specific to one of more complementary A, C, G, T, or U, or complementary to a purine (i.e., A or G, or variant thereof) or pyrimidine (i.e., C, T, or U, or variant thereof). In some examples, a splinter oligo may be single-stranded or double stranded, in some cases, a splinter oligo molecule is circular. A splinter oligo may comprise one or more modified nucleotides, such as methylated nucleotides and nucleotide analogs. The splinter oligo may be added to a sample unbound or bound to another nucleic acid. In same cases, the splinter oligo is bound to a nucleic acid of a binding reagent.

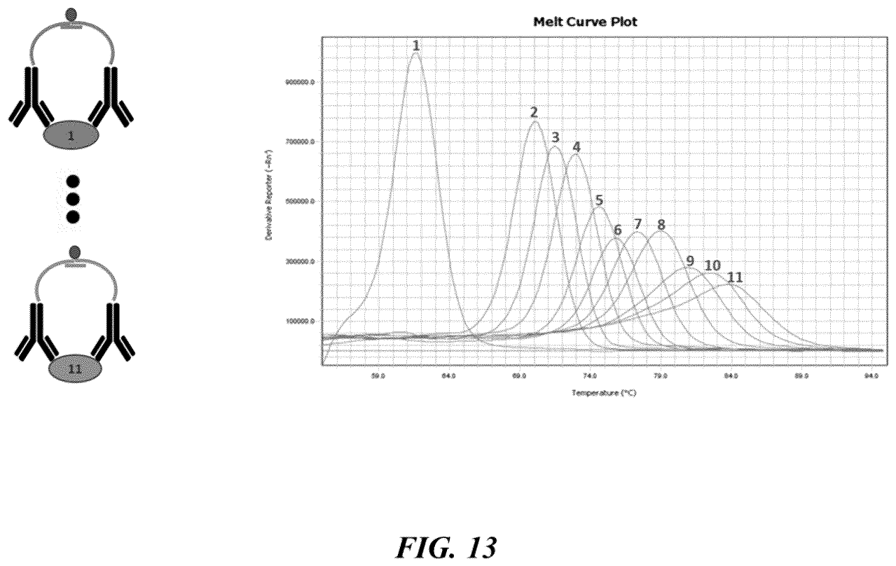

In an example, multiple binding reagents may be used to determine the presence of and quantify multiple analytes for use in a multiplexed analysis. In some cases, the PLA and dPCR methods described herein may include using at least 2, 4, 6, 8, 10, 12, 14, 16, 18, 20, 24, 28, 32, 26, 40, 50, 60, or more different binding reagents. In an example the PLA and dPCR methods may use between about 4 to about 50 different binding reagents in order to detect between at least 2 and about 25 different analytes in a multiplexed analysis. The methods may involve generating a denaturation profile of amplified PLA products in order to determine the presence and quantification of at least 2 to about 50 different analytes. In some cases, the denaturation profiles of amplified PLA products are usable to identify a proximal relationship of two different analytes to each other. In some cases, the denaturation profiles are derived from signals from one or more intercalating dyes. FIG. 13 schematically illustrates an example using PLA and dPCR with digital melt analysis with an intercalating dye to detect eleven different analytes.

### Systems and Devices for Identifying Analytes

In an aspect, the present disclosure provides a system for identifying analytes. The system may include a detection unit and one or more processors. The detection unit may be configured to collect and process or may collect and process signals for the identification of an analyte. The one or more processors may be operatively coupled to the detection units. The one or more processors may be programmed or otherwise configured to contact a binding reagent with the analyte. The binding reagent may include an analyte-specific binding probe and a nucleic acid molecule. The one or more processors may be further programmed or configured to denature at least a portion of the nucleic acid molecule or derivates thereof and direct the detection unit to collect signals indicative of the denaturing of the nucleic acid molecule. The signals indicative of the denaturing of the nucleic acid molecule may be used to identify the analyte.

The system may be configured to implement or may implement any of the methods described elsewhere herein. The system may use any of the devices, reagents, or components described elsewhere herein.

The system may further include a support. The support may be configured to immobilize the analyte. The analyte may be provided in a solution or coupled to a support. The analyte may be coupled to a support via a capture agent, covalently immobilized to the support (e.g., via a linker molecule), physically absorbed to a support, or any combination thereof. In an example, an analyte is provided in solution and the method includes coupling the analyte to a support. The analyte in solution may be brought into contact with a support comprising one or more capture agents. The capture agents may be immobilized on the support and may be usable for immobilizing the analyte onto the support. The capture agent may be a ligand, nucleic acid molecule, antibody, or any other molecule capable of binding to the analyte. The capture agent may have specificity for the analyte or may be a universal capture agent capable of binding multiple different analytes or different types of analytes (e.g., proteins, nucleic acid molecules, metabolites, etc.). In an example, the capture agent comprises an antibody and the analyte comprises a protein. In another example, the capture agent comprises an aptamer and the analyte comprises a protein, nucleic acid molecule, cell, metabolite, or other biological molecule. The capture agent may comprise a binding region that is specific to an analyte. Alternatively, or in addition to, the capture agent may comprise a binding region that is specific to a domain of an analyte, permitting the capture agent to bind to multiple different analytes.

The one or more processors may be configured or otherwise programed to contact the analyte with the binding reagent. The binding reagent may comprise a binding probe and a nucleic acid molecule. The binding probe may comprise a ligand, nucleic acid molecule, antibody, or any other molecule capable of binding to the analyte. In an example, the binding probe is an antibody and the binding reagent comprises an antibody. In another example, the binding probe is an aptamer and the binding reagent comprises an aptamer. The binding probe may have specificity for an analyte or portion of an analyte. The binding probe may not have specificity for another analyte or another portion of an analyte, thus, permitting the binding reagent to bind to a single analyte or type of analyte. The binding probe may permit reversible binding of the binding reagent to the analyte. Alternatively, or in addition, the binding probe may irreversibly bind the binding reagent to the analyte.

A binding reagent may include a nucleic acid molecule and a different binding reagent may include a different nucleic acid molecule. The nucleic acid molecule may be usable for detecting or identifying the analyte. For example, a first binding reagent may comprise a first nucleic acid molecule and may couple to a first analyte. A second binding reagent may comprise a second nucleic acid molecule and may couple to a second analyte. The first nucleic acid molecule and the second nucleic acid molecule may be different molecules. The differences in the first nucleic acid molecule and the second nucleic acid molecule may be detectable and, therefore, usable for identifying and distinguishing the first analyte from the second analyte. A sample may be contact with at least 1, 2, 3, 4, 5, 6, 8, 10, 15, 20, 30, 40, 50, or more binding reagents each coupled to at least 1, 2, 3, 4, 5, 6, 8, 10, 15, 20, 30, 40, 50, or more different nucleic acid molecules. A nucleic acid molecule may include a molecule or sequence (e.g., identifier or barcode) that is usable for identifying a given analyte.

The system may be configured to or may otherwise identify an analyte via identification of the nucleic acid molecule. Identification of the nucleic acid molecule may permit detection, identification, quantification, or any combination thereof of an analyte(s). The binding reagent (e.g., binding probe coupled to a nucleic acid molecule) may be analyzed to identify the nucleic acid molecule. Alternatively, or in addition to, the nucleic acid molecule or a portion of the nucleic acid molecule may be removed or cleaved from the binding reagent prior to identification or detection. The nucleic acid molecule may be reversibly coupled to the binding probe or may be irreversibly coupled to the binding probe. In an example, the nucleic acid molecule is reversibly coupled to the binding probe. In another example, the nucleic acid molecule is irreversibly coupled to the binding probe. A reversibly coupled nucleic acid molecule may be coupled to the binding probe by disulfide bonds, ligand binding, or other reversible bond. The reversibly coupled nucleic acid molecule may be decoupled or disassociate by contacting the binding reagent with a reducing agent, molecule with higher affinity for the binding probe, or any combination thereof. Alternatively, or in addition to, the nucleic acid molecule may be irreversibly coupled to the binding probe (e.g., covalently coupled). The nucleic acid molecule may be cleaved prior to analysis of the nucleic acid molecule. For example, the nucleic acid may include a consensus sequence for a restriction enzyme and the restriction enzyme may cleave the nucleic acid from the binding probe. Decoupling or cleaving of the nucleic acid molecule from the binding probe may permit the nucleic acid molecule to enter the solution phase while the analyte and binding probe remain immobilized on the support.

The system may further include a microfluidic device. The microfluidic device may include a plurality of partitions (e.g., an array of partitions). The nucleic acid molecule may be one of a plurality of nucleic acid molecule and the system may provide the plurality of nucleic acid molecules the microfluidic device for partitioning. The system may be further configured to amplify the nucleic acid molecules and subject the nucleic acid molecules to denaturation conditions in the partitions.

The microfluidic devices of the present disclosure may be consumable devices (e.g., designed for a single use, such as analysis or processing of a single sample) or reusable devices (e.g., designed for multiple uses, such as analysis or processing of multiple samples). The choices of materials for inclusion in the device may reflect whether the device will be used one or more times. For example, a consumable device may comprise materials that are less expensive than a reusable device. Similarly, manufacturing processes may be tailored to the use of the device. For example, a fabrication process for a consumable device may involve the production of less waste or involve lower cost manufacturing. A reusable device may be cleanable or sterilizable to facilitate analyses or processing of multiple samples using the same device. For example, a reusable device may comprise materials capable of withstanding high temperatures appropriate for sterilization. A consumable device may or may not comprise such materials.

The microfluidic device may include a fluid flow path. The fluid flow path may include one channel or multiple channels. The fluid flow path may include 1, 2, 3, 4, 5, 6, 8, 10, 12, 15, 20, 25, 30, 40, 50 or more channels. Each channel may be fluidically isolated from one another. Alternatively, or in addition to, the multiple channels may be in fluidic communication with one another. The channel may include a first end and a second end. The first end and second end may be connected to a single inlet port. A channel with a first end and second end connected to a single inlet port may be in a circular or looped configuration such that the fluid entering the channel through the inlet port may be directed through the first end and second end of the channel simultaneously. Alternatively, the first end and second end may be connected to different inlet ports. Alternatively, the first end may be connected to an inlet port and the second end may be connected to an outlet port. Alternatively, the first end may be connected to an inlet port and the second end may be a closed or dead end. The fluid flow path may include a plurality of partitions (e.g., chambers). The fluid flow path or the chambers may not include valves to stop or hinder fluid flow or to isolate the chamber(s).

The device may comprise a long dimension and a short dimension. The long dimension may be less than or equal to about 20 centimeters (cm), 15 cm, 10 cm, 8 cm, 6 cm, 5 cm, 4 cm, 3 cm, 2 cm, 1 cm, or less. The short dimension of the device may be less than or equal to about 10 cm, 8 cm, 6 cm, 5 cm, 4 cm, 3 cm, 2 cm, 1 cm, 0.5 cm, or less. In an example, the dimensions of the device (e.g., microfluidic device) are about 7.5 cm by 2.5 cm. The channel may be substantially parallel to the long dimension of the microfluidic device. Alternatively, or in addition to, the channel may be substantially perpendicular to the long dimension of the microfluidic device (e.g., parallel to the short dimension of the device). Alternatively, or in addition to, the channel may be neither substantially parallel nor substantially perpendicular to the long dimension of the microfluidic device. The angle between the channel and the long dimension of the microfluidic device may be at least about 5°, 10°, 15°, 20°, 30°, 40°, 50°, 60°, 70°, or 90. In an example, the channel is a single long channel. Alternatively, or in addition to, the channel may have bends, curves, or angles. The channel may have a long dimension that is less than or equal to about 100 millimeters (mm), 75 mm, 50 mm, 40 mm, 30 mm, 20 mm, 10 mm, 8 mm, 6 mm, 4 mm, 2 mm, or less. The length of the channel may be bounded by the external length or width of the microfluidic device. The channel may have a depth of less than or equal to about 500 micrometers (μm), 250 μm, 100 μm, 80 μm, 60 μm, 30 μm, 20 μm, 10 μm, or less. The channel may have a cross-sectional dimension (e.g., width or diameter) of less than or equal to about 500 μm, 250 μm, 100 μm, 75 μm, 50 μm, 40 μm, 30 μm, 20 μm, 10 μm, or less.

In some examples, the cross-sectional dimensions of the channel may be about 100 μm wide by about 100 μm deep. In some examples, the cross-sectional dimensions of the channel may be about 100 μm wide by about 80 μm deep. In some examples, the cross-sectional dimensions of the channel may be about 100 μm wide by about 60 μm deep. In some examples, the cross-sectional dimensions of the channel may be about 100 μm wide by about 40 μm deep. In some examples, the cross-sectional dimensions of the channel may be about 100 μm wide by about 20 μm deep. In some examples, the cross-sectional dimensions of the channel may be about 100 μm wide by about 10 μm deep. In some examples, the cross-sectional dimensions of the channel may be about 80 μm wide by about 100 μm deep. In some examples, the cross-sectional dimensions of the channel may be about 60 μm wide by about 100 μm deep. In some examples, the cross-sectional dimensions of the channel may be about 40 μm wide by about 100 μm deep. In some examples, the cross-sectional dimensions of the channel may be about 20 μm wide by about 100 μm deep. In some examples, the cross-sectional dimensions of the channel may be about 10 μm wide by about 100 μm deep. In some examples, the cross-sectional dimensions of the channel may be about 80 μm wide by about 80 μm deep. In some examples, the cross-sectional dimensions of the channel may be about 60 μm wide by about 60 μm deep. In some examples, the cross-sectional dimensions of the channel may be about 40 μm wide by about 40 μm deep. In some examples, the cross-sectional dimensions of the channel may be about 20 μm wide by about 20 μm deep. In some examples, the cross-sectional dimensions of the channel may be about 10 μm wide by about 10 μm deep.

The cross-sectional shape of the channel may be any suitable cross-sectional shape including, but not limited to, circular, oval, triangular, square, or rectangular. The cross-sectional area of the channel may be constant along the length of the channel. Alternatively, or in addition to, the cross-sectional area of the channel may vary along the length of the channel. The cross-sectional area of the channel may vary from about 50% to 150%, 60% to 125%, 70% to 120%, 80% to 115%, 90% to 110%, 95% to 100%, or 98% to 102%. The cross-sectional area of the channel may be less than or equal to about 10,000 micrometers squared (μm²), 7,500 μm², 5,000 μm², 2,500 μm², 1,000 μm², 750 μm², 500 μm², 400 μm², 300 μm², 200 μm², 100 μm², or less.

The channel may have a single inlet or multiple inlets. The inlet(s) may have the same diameter or they may have different diameters. The inlet(s) may have diameters less than or equal to about 2.5 millimeters (mm), 2 mm, 1.5 mm, 1 mm, 0.5 mm, or less.

The device may include a plurality of chambers. The plurality of chambers may be an array of chambers. The device may include a single array of chambers or multiple arrays of chambers, with each array of chambers fluidically isolated from the other arrays. The array of chambers may be arranged in a row, in a grid configuration, in an alternating pattern, or in any other configuration. The microfluidic device may have at least 1, 2, 3, 4, 5, 10, 15, 20, 30, 40, 50, or more arrays of chambers. The arrays of chambers may be identical of the arrays of chambers may be different (e.g., have a different number or configuration of chambers). The arrays of chambers may all have the same external dimension (i.e., the length and width of the array of chambers that encompasses all features of the array of chambers) or the arrays of chambers may have different external dimensions. An array of chambers may have a width of less than or equal to about 100 mm, 75 mm, 50 mm, 40 mm, 30 mm, 20 mm, 10 mm, 8 mm, 6 mm, 4 mm, 2 mm, 1 mm, or less. The array of chambers may have a length of greater than or equal to about 50 mm, 40 mm, 30 mm, 20 mm, 10 mm, 8 mm, 6 mm, 4 mm, 2 mm, 1 mm, or less. In an example, the width of an array may be from about 1 mm to 100 mm or from about 10 mm to 50 mm. In an example, the length of an array may be from about 1 mm to 50 mm or from about 5 mm to 20 mm.

The array of chambers may have greater than or equal to about 1,000 chambers, 5,000 chambers, 10,000 chambers, 20,000 chambers, 30,000 chambers, 40,000 chambers, 50,000 chambers, 100,000 chambers, or more. In an example, the microfluidic device may have from about 10,000 to 30,000 chambers. In another example, the microfluidic device may have from about 15,000 to 25,000 chambers. The chambers may be cylindrical in shape, hemispherical in shape, or a combination of cylindrical and hemispherical in shape. Alternatively, or in addition to, the chambers may be cubic in shape. The chambers may have a cross-sectional dimension of less than or equal to about 500 μm, 250 μm, 100 μm, 80 μm, 60 μm, 30 μm, 15 μm, or less. In an example, the chamber has a cross-sectional dimension (e.g., diameter or side length) that is less than or equal to about 250 μm. In another example, the chamber has a cross-sectional dimension (e.g., diameter or side length) that is less than or equal to about 100 μm. In another example, the chamber has a cross-sectional dimension (e.g., diameter or side length) that is less than or equal to about 50 μm.

The depth of the chambers may be less than or equal to about 500 μm, 250 μm, 100 μm, 80 μm, 60 μm, 30 μm, 15 μm, or less. In an example, the chambers may have a cross-sectional dimension of about 30 μm and a depth of about 100 μm. In another example, the chambers may have a cross-sectional dimension of about 35 μm and a depth of about 80 μm. In another example, the chambers may have a cross-sectional dimension of about 40 μm and a depth of about 70 μm. In another example, the chambers may have a cross-sectional dimension of about 50 μm and a depth of about 60 μm. In another example, the chambers may have a cross-sectional dimension of about 60 μm and a depth of about 40 μm. In another example, the chambers may have a cross-sectional dimension of about 80 μm and a depth of about 35 μm. In another example, the chambers may have a cross-sectional dimension of about 100 μm and a depth of about 30 μm. In another example, the chambers and the channel have the same depth. In an alternative embodiment, the chambers and the channel have different depths.

The chambers may have any volume. The chambers may have the same volume or the volume may vary across the microfluidic device. The chambers may have a volume of less than or equal to about 1000 picoliters (pL), 900 pL, 800 pL, 700 pL, 600 pL, 500 pL, 400 pL, 300 pL, 200 pL, 100 pL, 75 pL, 50 pL, 25 pL, or less picoliters. The chambers may have a volume from about 25 pL to 50 pL, 25 pL to 75 pL, 25 pL to 100 pL, 25 pL to 200 pL, 25 pL to 300 pL, 25 pL to 400 pL, 25 pL to 500 pL, 25 pL to 600 pL, 25 pL to 700 pL, 25 pL to 800 pL, 25 pL to 900 pL, or 25 pL to 1000 pL. In an example, the chamber(s) have a volume of less than or equal to 500 pL. In another example, the chambers have a volume of less than or equal to about 250 pL. In another example, the chambers have a volume of less than or equal to about 100 pL.

The volume of channel may be less than, equal to, or greater than the total volume of the chambers. In an example, the volume of the channel is less than the total volume of the chambers. The volume of the channel may be less than or equal to 95%, 90%, 80%, 70%, 60%, 50%, 40%, 30%, 20%, 10%, or less than the total volume of the chambers.

The device may further include a siphon aperture disposed between the channel and the chamber. The siphon aperture may be one of a plurality of siphon apertures connecting the channel to a plurality of chambers. The siphon aperture may be configured to provide fluid communication between the channel and the chamber. The lengths of the siphon apertures may constant or may vary across the device (e.g., microfluidic device). The siphon apertures may have a long dimension that is less than or equal to about 150 μm, 100 μm, 50 μm, 25 μm, 10 μm, 5 μm, or less. The depth of the siphon aperture may be less than or equal to about 50 μm, 25 μm, 10 μm, 5 μm, or less. The siphon apertures may have a cross-sectional dimension of less than or equal to about 50 μm, 40 μm, 30 μm, 20 μm, 10 μm, 5 μm, or less.

The cross-sectional shape of the siphon aperture may be any suitable cross-sectional shape including, but not limited to, circular, oval, triangular, square, or rectangular. The cross-sectional area of the siphon aperture may be constant along the length of the siphon aperture. Alternatively, or in addition to, the cross-sectional area of the siphon aperture may vary along the length of the siphon aperture. The cross-sectional area of the siphon aperture may be greater at the connection to the channel than the cross-sectional area of the siphon aperture at the connection to the chamber. Alternatively, the cross-sectional area of the siphon aperture at the connection to the chamber may be greater than the cross-sectional area of the siphon aperture at the connection to the channel. The cross-sectional area of the siphon aperture may vary from about 50% to 150%, 60% to 125%, 70% to 120%, 80% to 115%, 90% to 110%, 95% to 100%, or 98% to 102%. The cross-sectional area of the siphon aperture may be less than or equal to about 2,500 μm², 1,000 μm², 750 μm², 500 μm², 250 μm², 100 μm², 75 μm², 50 μm², 25 μm², or less. The cross-sectional area of the siphon aperture at the connection to the channel may be less than or equal to the cross-sectional area of the channel. The cross-sectional area of the siphon aperture at the connection to the channel may be less than or equal to about 98%, 95%, 90%, 85%, 80%, 75%, 70%, 60%, 50%, 40%, 30%, 20%, 10%, 5%, 1%, 0.5%, or less of the cross-sectional area of the channel. The siphon apertures may be substantially perpendicular to the channel. Alternatively, or in addition to, the siphon apertures are not substantially perpendicular to the channel. An angle between the siphon apertures and the channel may be at least about 5°, 10°, 15°, 20°, 30°, 40°, 50°, 60°, 70°, or 90°.

The microfluidic device may be configured to permit pressurized off-gassing or degassing of the channel, chamber, siphon aperture, or any combination thereof. Pressurized off-gassing or degassing may be provided by a film or membrane configured to permit pressurized off-gassing or degassing. Alternatively, or in addition to, pressurized off-gassing or degassing may be provided by a second channel (e.g., off-gas channel) disposed adjacent to the chamber, channels, or both. The second channel may permit pressurized off-gassing or degassing above a pressure threshold. The film or membrane may be permeable to gas above a pressure threshold. The film or membrane may not be permeable to (e.g., is impermeable or substantially impermeable to) liquids such as, but not limited to, aqueous fluids, oils, or other solvents. The channel, the chamber, the siphon aperture, or any combination thereof may comprise the film or membrane. In an example, the chamber comprises the gas permeable film or membrane and the channel or siphon aperture does not comprise the gas permeable film or membrane. In another example, the chamber and siphon aperture comprises the gas permeable film or membrane and the channel does not comprise the gas permeable film or membrane. In another example, the chamber, channel, and siphon aperture comprise the gas permeable film or membrane.

The film or membrane may be a thin file. The film or membrane may be a polymer. The film may be a thermoplastic film or membrane. The film or membrane may not comprise an elastomeric material. The gas permeable film or membrane may cover the fluid flow path, the channel, the chamber, or any combination thereof. In an example, the gas permeable film or membrane covers the chamber. In another example, the gas permeable film or membrane covers the chamber and the channel. The gas permeability of the film may be induced by elevated pressures. The thickness of the film or membrane may be less than or equal to about 500 micrometers (μm), 250 μm, 200 μm, 150 μm, 100 μm, 75 μm, 50 μm, 25 μm, or less. In an example, the film or membrane has a thickness of less than or equal to about 100 μm. In another example, the film or membrane has a thickness of less than or equal to about 50 μm. In another example, the film or membrane has a thickness of less than or equal to about 25 μm. The thickness of the film or membrane may be from about 0.1 μm to about 200 μm, 0.5 μm to 150 μm, or 25 μm to 100 μm. In an example, the thickness of the film or membrane is from about 25 μm to 100 μm. The thickness of the film may be selected by manufacturability of the film, the air permeability of the film, the volume of each chamber or partition to be out-gassed, the available pressure, or the time to complete the partitioning or digitizing process.

The film or second channel may be configured to employee different permeability characteristics under different applied pressure differentials. For example, the thin film or second channel may be gas impermeable at a first pressure differential (e.g., low pressure) and at least partially gas permeable at a second pressure differential (e.g., high pressure). The first pressure differential (e.g., low pressure differential) may be less than or equal to about 8 pounds per square inch (psi), 6 psi, 4 psi, 2 psi, 1 psi, or less. In an example, the film or membrane is substantially impermeable to gas at a pressure differential of less than 4 psi. The second pressure differential (e.g., high pressure differential) may be greater than or equal to about 1 psi, 2 psi, 4 psi, 6 psi, 8 psi, 10 psi, 12 psi 14 psi, 16 psi, 20 psi, or more. In an example, the film or membrane is substantially gas permeable at a pressure of greater than or equal to 4 psi.

The system may include a holder configured to receive or hold the microfluidic device. The holder may be a shelf, receptacle, or stage for holding the device. In an example, the holder is a transfer stage. The transfer stage may be configured input the microfluidic device, hold the microfluidic device, and output the microfluidic device. The microfluidic device may be any device described elsewhere herein. The transfer stage may be stationary in one or more coordinates. Alternatively, or in addition to, the transfer stage may be capable of moving in the X-direction, Y-direction, Z-direction, or any combination thereof. The transfer stage may be capable of holding a single microfluidic device. Alternatively, or in addition to, the transfer stage may be capable of holding at least 2, 3, 4, 5, 6, 7, 8, 9, 10, or more microfluidic devices.

The system may include a processing unit. The processing unit may include a pneumatic module, vacuum module, or any combination thereof. The processing unit may be configured to amplify the nucleic acid molecules in the plurality of partitions or chambers. The processing unit may be configured to be in fluid communication with the inlet port(s) of the microfluidic device. The processing unit may have multiple connection points capable of connecting to multiple inlet port(s). The processing unit may be able to fill, backfill, and partition a single array of chambers at a time or multiple arrays of chambers in tandem. The processing unit may be a pneumatic module combined with a vacuum module. The processing unit may provide increased pressure to the microfluidic device or provide vacuum to the microfluidic device for pressurized off-gassing or degassing.

The system may further comprise a thermal unit. The thermal module may be configured to be in thermal communication with the chambers of the microfluidic devices. The thermal unit may be configured to control the temperature of a single array of chambers or to control the temperature of multiple arrays of chambers to permit thermal denaturation of the nucleic acid molecules. An array of chambers may be individually addressable by the thermal module. For example, thermal module may perform the same thermal program across all arrays of chambers or may perform different thermal programs with different arrays of chambers. The thermal unit may be in thermal communication with the microfluidic device or the chambers of the microfluidic device. The thermal unit may heat or cool the microfluidic device. One or more surfaces of the microfluidic device may be in direct contact with the thermal unit. Alternately, or in addition to, a thermally conductive material may be disposed between the thermal module and the microfluidic device. The thermal unit may maintain the temperature across a surface of the microfluidic device such that the variation is less than or equal to about 2° C., 1.5° C., 1° C., 0.9° C., 0.8° C., 0.7° C., 0.6° C., 0.5° C., 0.4° C., 0.3° C., 0.2° C., 0.1° C., or less. The thermal unit may maintain a temperature of a surface of the microfluidic device that is within about plus or minus 0.5° C., 0.4° C., 0.3° C., 0.2° C., 0.1° C., 0.05° C., or closer to a temperature set point.

The system may further include a detection unit. The detection module may provide electronic or optical detection. In an example, the detection unit is an optical unit providing optical detection. The optical unit may be configured to emit and detect multiple wavelengths of light. Emission wavelengths may correspond to the excitation wavelengths of the indicator and amplification probes used. The emitted light may include wavelengths with a maximum intensity around about 450 nm, 500 nm, 525 nm, 550 nm, 575 nm, 600 nm, 625 nm, 650 nm, 675 nm, 700 nm, or any combination thereof. Detected light may include wavelengths with a maximum intensity around about 500 nm, 525 nm, 550 nm, 575 nm, 600 nm, 625 nm, 650 nm, 675 nm, 700 nm, or any combination thereof. The optical unit may be configured to emit more than one, two, three, four, or more wavelengths of light. The optical unit may be configured to detect more than one, two, three, four, or more wavelengths of light. One emitted wavelength of light may correspond to the excitation wavelength of an indicator molecule. Another emitted wavelength of light may correspond to the excitation wavelength of an amplification probe. One detected wavelength of light may correspond to the emission wavelength of an indicator molecule. Another detected wavelength of light may correspond to an amplification probe used to detect a reaction within the chambers. The optical unit may be configured to image sections of an array of chambers. Alternatively, or in addition to, the optical unit may image an entire array of chambers in a single image. In an example, the optical unit is configured to take video of the device.

The system may further include a robotic arm. The robotic arm may move, alter, or arrange a position of the microfluidic device. Alternatively, or in addition to, the robotic arm may arrange or move other components of the system (e.g., processing unit or detection unit). The detection unit may include a camera (e.g., a complementary metal oxide semiconductor (CMOS) camera) and filter cubes. The filter cubes may alter or modify the wavelength of excitation light or the wavelength of light detected by the camera. The processing unit may comprise a manifold (e.g., pneumatic manifold) or one or more pumps. The manifold may be in an upright position such that the manifold does not contact the microfluidic device. The upright position may be used when loading or imaging the microfluidic device. The manifold may be in a downward position such that the manifold contacts the microfluidic device. The manifold may be used to load fluids (e.g., samples and reagents) into the microfluidic device. The manifold may apply a pressure to the microfluidic device to hold the device in place or to prevent warping, bending, or other stresses during use. In an example, the manifold applies a downward pressure and holds the microfluidic device against the thermal unit.

The system may further include one or more computer processors. The one or more computer processors may be operatively coupled to the processing unit, holder, thermal unit, detection unit, robotic arm, or any combination thereof. In an example, the one or more computer processors is operatively coupled to the processing unit. The one or more computer processors may be individually or collectively programmed to direct the processing unit to partition the nucleic acid molecules, amplify the nucleic acid molecules, denature the nucleic acid molecule, or any combination thereof. The one or more computer processors may be individually or collectively programmed or otherwise configured to direct the detection unit to collect signals indicative of denaturation of the nucleic acid molecules. The one or more computer processors may be individually or collectively programmed or otherwise configured to generate a denaturation profile of the nucleic acid molecule, use the denaturation profile to identify or quantify the analyte, or any combination thereof.

### Computer Systems

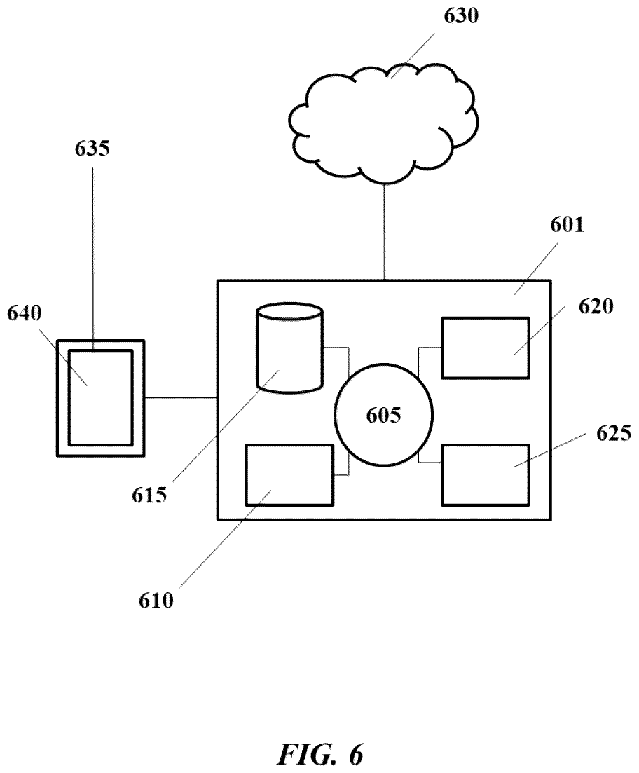

The present disclosure provides computer systems that are programmed to implement methods of the disclosure. FIG. 6 shows a computer system 601 that is programmed or otherwise configured to implement the methods described elsewhere for analysis of analytes. The computer system 601 can regulate various aspects of processing and detection of analytes of the present disclosure, such as, for example, contacting an analyte with a binding reagent, denaturing the nucleic acid molecule, detecting signals associated with nucleic acid denaturation, or processing the signals to identify or quantify the analyte. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet or extranet, or an intranet or extranet that is in communication with the Internet. The network 630 in some cases is a telecommunication or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., cell phone or laptop). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine-executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, system operating parameters, system status, or analysis results. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, process denaturation signals to provide identification or quantification of an analyte.

### EXAMPLES

### Example 1: Interleukin 6 Standard Curve

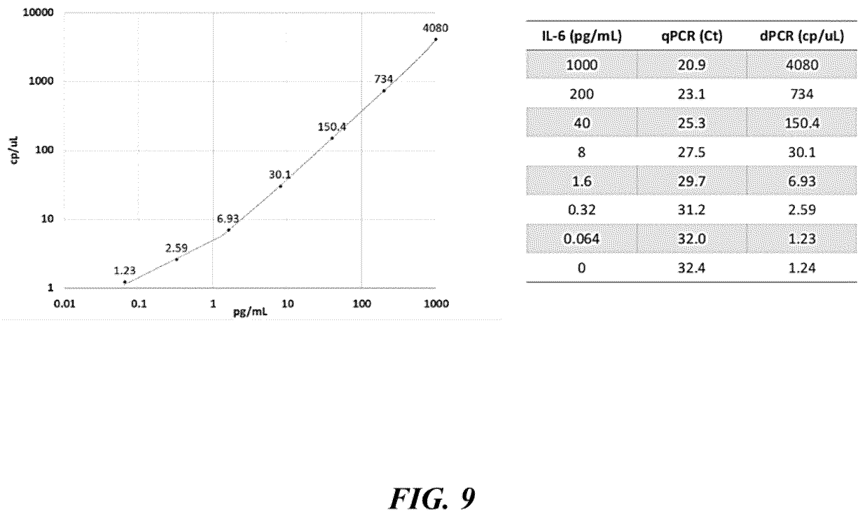

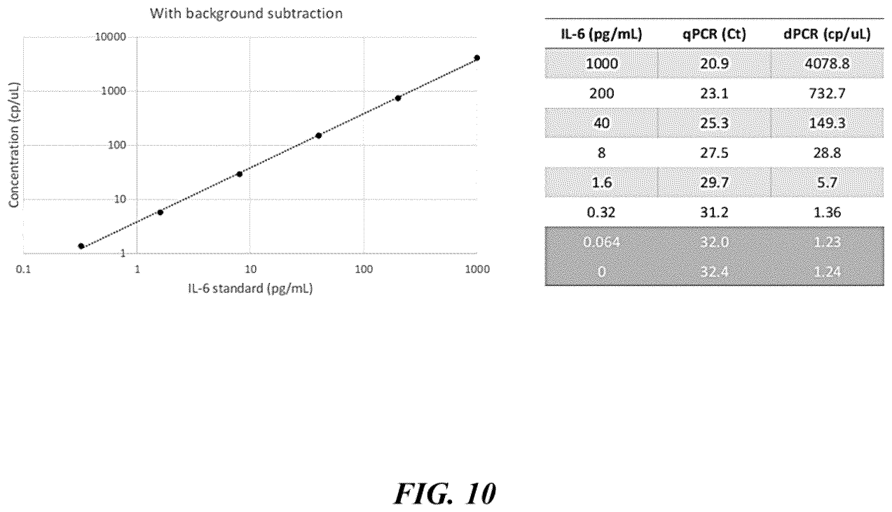

In an example, a protein (e.g., interleukin 6) may be quantified to generate a standard curve useful for subsequent protein quantification, as illustrated in FIG. 9. Samples containing select amounts of a protein (e.g., interleukin 6) ranging from 0 to 1000 picogram per milliliter (pg/mL) are mixed with ssDNA antibody binding reagents, splinter oligos, a master mix of PLA and dPCR reagents, and ligase to create a PLA reaction mixture. The PLA reaction is performed and 1 μL of each sample is loaded onto multiwell plates for dPCR thermocycling and analysis. The resulting concentration output in copies per microliter is plotted as a function of interleukin 6 amounts to generate a standard curve, as illustrated in FIG. 9. Output concentration values range from 1.23 cp/μL to 4078.8 cp/μL. To improve the linearity of the graph data, the background signal, as determined from samples having no interleukin 6, is subtracted from the interleukin 6 containing sample signals to generate another standard curve, as illustrated in FIG. 10. The assay is able to detect as little as 320 attograms of interleukin 6 per mL within the linear range of the normalized standard curve.

### Example 2: Troponin Standard Curve

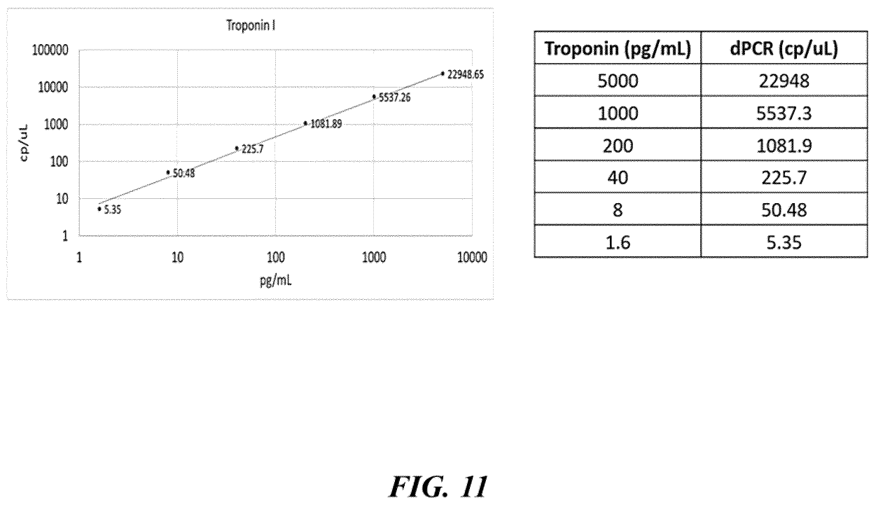

In another example, Troponin protein is used to generate a standard curve for protein quantification, as illustrated in FIG. 11. Samples containing select amounts of troponin ranging from 0 to 5000 pg/mL are mixed with ssDNA antibody binding reagents, splinter oligos, a master mix of PLA and dPCR reagents, and ligase to create a PLA reaction mixture. The PLA reaction is allowed to occur and 1 μL of each sample is loaded onto multiwell plates for dPCR thermocycling and analysis. The resulting concentration, output in copies per microliter, is plotted against the select troponin amounts to generate a standard curve, as illustrated in FIG. 11. Output concentration values range from 5.35 cp/μL to 22948 cp/μL. The assay is able to detect as little as 1.6 picograms of troponin per mL within the linear range of the normalized standard curve. In both of the previous examples, it is shown that PLA is an effective method for analyte detection upstream of a qPCR workflow and suitable for analyte quantification.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for identifying an analyte, comprising:
(a) contacting a binding reagent with said analyte, which binding reagent comprises:
(i) a binding probe having binding specificity for said analyte, and (ii) a nucleic acid molecule;
(b) denaturing at least a portion of said nucleic acid molecule or derivative thereof; and
(c) detecting signals indicative of said denaturing to identify said analyte.

2. The method of claim 1, wherein said analyte is coupled to a support.

3. The method of claim 2, wherein said analyte is coupled to said support via a capture agent immobilized on said support.

4. The method of claim 3, wherein said capture agent is an antibody.

5. The method of claim 1, wherein said binding reagent is an antibody.

6. The method of claim 1, wherein said analyte is a protein.

7. The method of claim 1, wherein said analyte is one of a plurality of analytes in a sample, and wherein said binding reagent is one of a plurality of binding reagents.

8. The method of claim 7, further comprising contacting an additional binding reagent with an additional analyte, which said additional binding reagent comprises: (i) an additional binding probe having binding specificity for said additional analyte, and (ii) an additional nucleic acid molecule.

9. The method of claim 8, wherein said additional nucleic acid molecule is different than said nucleic acid molecule.

10. The method of claim 8, further comprising denaturing said additional nucleic acid molecule or derivative thereof.

11. The method of claim 10, further comprising detecting additional signals indicative of said denaturing said additional nucleic acid or derivative thereof to identify said additional analyte.

12. The method of claim 7, further comprising partitioning said plurality of binding reagents into a plurality of partitions.

13. The method of claim 12, further comprising using a microfluidic device to partition said plurality of binding reagents into said plurality of partitions.

14. The method of claim 12, further comprising imaging one or more partitions of said plurality of partitions to detect said signals.

15. The method of claim 14, further comprising determining a number of partitions which contain said nucleic acid molecule, wherein said number of partitions which contain said nucleic acid molecule are used to quantify said analyte.

16. The method of claim 1, wherein said nucleic acid molecule is reversibly coupled to said binding probe.

17. The method of claim 16, further comprising, prior to (b), decoupling said nucleic acid molecule from said binding probe.

18. The method of claim 1, further comprising, prior to (b), decoupling a portion of said nucleic acid molecule from said binding probe.

19. The method of claim 1, wherein, subsequent to (a), said binding reagent is coupled to said analyte.

20. The method of claim 19, further comprising removing uncoupled binding reagent by washing.

21. The method of claim 1, further comprising subjecting said nucleic acid molecule to controlled heating to denature said nucleic acid molecule.

22. The method of claim 1, further comprising, prior to (b), amplifying said nucleic acid molecule.

23. The method of claim 1, further comprising processing said signals to generate a denaturation profile, which denaturation profile is used to identify said analyte.

24. The method of claim 1, wherein said nucleic acid molecule comprises an intercalating dye from which said signals are derived.

25. The method of claim 1, wherein said signals are optical signals.

26. The method of claim 1, wherein (a) further comprises contacting an additional binding reagent with said analyte, which additional binding reagent comprises: (i) an additional binding probe having binding specificity for said analyte, and (ii) an additional nucleic acid molecule.

27. The method of claim 26, further comprising contacting said nucleic acid molecule and said additional nucleic acid molecule with a splinter oligo to couple said nucleic acid molecule with said additional nucleic acid molecule.

28. The method of claim 27, further comprising ligating said nucleic acid molecule and said additional nucleic acid molecule to generate a ligated nucleic acid molecule.

29. The method of claim 28, further comprising, prior to (b), amplifying only said ligated nucleic acid molecule.

30. A system for identifying an analyte, comprising:
a detection unit configured to collect and process signals for identification of said analyte; and
one or more processors operatively coupled to said detection unit, wherein said one or more processors are individually or collectively programmed or otherwise configured to:
(i) contact a binding reagent with said analyte, which binding reagent comprises (a) a binding probe having binding specificity for said analyte, and (b) a nucleic acid molecule;
(ii) denature at least a portion of said nucleic acid molecule or derivative thereof; and
(iii) direct said detection unit to detect signals indicative of denaturing of said nucleic acid molecule to identify said analyte.

31. The system of claim 30, further comprising a support configured to couple to said analyte.

32. The system of claim 31, wherein said support comprises a capture agent immobilized on said support, and wherein said capture agent is configured to couple to said analyte.

33. The system of claim 32, wherein said capture agent is an antibody.

34. The system of claim 30, wherein said binding reagent is an antibody.

35. The system of claim 30, wherein said analyte is a protein.

36. The system of claim 30, further comprising a microfluidic device comprising a plurality of partitions.

37. The system of claim 36, wherein said plurality of partitions are configured to partition a mixture comprising said binding reagent.

38. The system of claim 37, wherein said detection unit is configured to image one or more partitions of said plurality of partitions.

39. The system of claim 30, further comprising a processing unit configured to amplify said nucleic acid molecule.

40. The system of claim 30, further comprising a heating unit configured for controlled heating of said nucleic acid molecule to denature said nucleic acid molecule.

41. The system of claim 30, wherein said one or more processors are individually or collectively programmed or otherwise configured to generate a denaturation profile of said nucleic acid molecule, which denaturation profile is usable to identify said analyte.
